(19) United States
(12) Patent Application Publication
(10) Pub. No.: US 2025/0339544 A1
(43) Pub. Date: Nov. 6, 2025

(54) COMPOSITION, METHODS AND USES

(71) Applicant: INFLANOVA AB, Stockholm (SE)

(72) Inventors: Johan FROSTEGÅRD, Stockholm (SE); Shailesh Kumar SAMAL, Stockholm (SE); Pritam Kumar PANDA, Stockholm (SE)

(73) Assignee: INFLANOVA AB, Stockholm (SE)

(21) Appl. No.: 18/864,303

(22) PCT Filed: May 9, 2023
(86) PCT No.: PCT/EP2023/062287

§ 371 Date: Nov. 8, 2024

(30) Foreign Application Priority Data
May 10, 2022 (GB) 2206807.6

(51) Int. Cl. A61K 47/64; C07K 16/18; G01N 33/569

(52) U.S. Cl. CPC A61K 47/64; C07K 16/18; G01N 33/56983; G01N 2333/165; G01N 2405/04; G01N 2469/20

(57) ABSTRACT

The present invention relates to: methods for predicting the prognosis of a virus infection in an individual; to methods for identifying an individual in need of anti-viral therapy; to related compositions; and to uses and treatment methods involving the compositions.

The present invention relates to: methods for predicting the prognosis of a virus infection in an individual; to methods for identifying an individual in need of anti-viral therapy; to related compositions; and to uses and treatment methods involving the compositions.

Viruses are found in almost every ecosystem on Earth and are the most numerous type of biological entity. Viruses cause viral infections in their hosts. These infections are not only harmful to human health but also to the health of various other animals, plants and microorganisms. Examples of common human diseases caused by viruses include respiratory diseases (such as the “common cold”), influenza, chickenpox, and cold sores. Many serious diseases such as rabies, Ebola virus disease, acquired immunodeficiency syndrome (AIDS), avian influenza, and severe acute respiratory syndrome (SARS) are also caused by viruses.

As can be seen from virus outbreaks over the last century, viruses represent a significant cause of disease and mortality. Viruses, and epidemics or pandemics caused by viruses, can have an enormous social and economic burden. In particular, the severe acute respiratory syndrome coronavirus 2 (SARS-CoV-2; Covid-19) has infected more than 100 million people and caused over 2 million deaths since January 2020 (World Health Organisation).

A wide range of treatments exist which seek to prevent and/or treat virus infections, and many also seek to lower the risk of virus spreading from host to host. Some viral infections will be cleared by the host without the need of treatment; however, vaccines and anti-viral drugs are often prescribed and extremely important for preventing and/or treating chronic and/or life-threatening virus infections.

Most vaccines and many anti-viral drugs are disease- or virus-specific, such that treatments used to prevent and/or treat infection caused by one type of virus may be ineffective to prevent and/or treat an infection caused by a different virus.

Clinical symptoms of virus infections in humans often overlap between different virus types and thus, it is not always clear which virus is responsible for causing the infection and thus which treatment to administer. For example, rhinovirus, Influenza virus, Respiratory Syncytial Virus and SARS-COV-2 have all been shown to cause cold-like symptoms (including coughing, headache and/or sore throat), and viruses such as Hepatitis A, Norovirus and Rotavirus have all been shown to cause gastrointestinal symptoms. Detection of virus infection using serology, culture and/or polymerase chain reaction (PCR) techniques can be used to differentiate between the virus, but this can be time-consuming, costly and labour intensive.

The same virus can be life threatening to one person but not in another. This depends on multiple factors such as age, genetics, underlying health conditions, and socio-economic factors. Some individuals are generally more susceptible to viruses than others (for example, the elderly, infants and immunocompromised individuals), and it can therefore be important to ensure that individuals at particular risk are identified early so they can receive appropriate prophylactic or therapeutic treatment. Advances in medical research has led to the concept of patient stratification, allowing the division of a general patient group into subgroups to steer therapeutic interventions and personalise treatments.

Further safe and effective treatments for preventing and/or treating virus infections are needed, particularly those which are effective against a range of virus types. In addition, further markers for predicting the severity and/or likely outcome of virus infection in individuals are also needed.

Against this background, the present inventors have surprisingly found that the endogenous molecule phosphorylcholine (“PC”) associates with viruses during infection in an individual. The inventors have also surprisingly identified that the level of anti-phosphorylcholine antibodies (“anti-PC antibodies”) in an individual correlate with the prognosis and/or severity of viral infection in an individual (and, notably, that anti-PC antibodies are lower among Covid-19 patients with severe disease, than among those with less severe disease).

Those findings are surprising, as there was previously no indication that PC and/or anti-PC antibodies were of any importance in virus infections, or that anti-PC antibodies could be used in the prevention or treatment of virus infections, or as a marker for determining disease severity.

Phosphorylcholine (PC) is a small molecule composed of a negatively charged phosphate group bonded to a small, positively charged choline group. It is a polar head group of many phospholipids found in cellular membranes and may also exist as a free molecule in multicellular organisms (including in humans). Anti-PC antibodies are natural antibodies that belong to the innate immune system. Natural antibodies have scavenging functions and are part of the first line defence against infections. Anti-PC antibodies can recognise PC epitopes formed in biological membranes during inflammation, for example immunogenic PC epitopes generated by oxidative and/or enzymatic modification of the membrane phospholipids. It is known that membranes containing immunogenic PC induce inflammation in other cells, and that this inflammation can be reduced and/or inhibited by anti-PC antibodies.

The inventors' findings therefore indicate that PC and anti-PC antibodies play an important role in virus infections, and that anti-PC antibodies have an anti-inflammatory role in virus infections. Those findings enable the development of further treatments for preventing and/or treating virus infections (particularly treatments effective against a range of virus types) and provide further biological markers for predicting the severity and/or likely outcome of virus infection in individuals.

Accordingly, in one aspect, the invention provides a protein complex comprising a carrier protein and phosphorylcholine, or an anti-phosphorylcholine antibody which binds specifically to a complex comprising a virus protein and phosphorylcholine, for use in treating and/or preventing a virus infection in an individual.

In a related aspect, the invention provides the use of a protein complex comprising a carrier protein and phosphorylcholine, or an anti-phosphorylcholine antibody which binds specifically to a complex comprising a virus protein and phosphorylcholine, in the manufacture of a medicament for use in treating and/or preventing a virus infection in an individual.

In a further related aspect, the invention provides a method for treating and/or preventing a virus infection in an individual, the method comprising administering to the individual an effective amount of a protein complex comprising a carrier protein and phosphorylcholine, or an anti-phosphorylcholine antibody which binds specifically to a complex comprising a virus protein and phosphorylcholine.

Thus, as discussed above and herein, in an embodiment, the uses and methods of the invention comprise the direct administration of anti-phosphorylcholine antibodies to the individual. Those anti-phosphorylcholine antibodies will bind specifically to a complex comprising a virus protein and phosphorylcholine, and thereby elicit an immune response to the virus, optionally leading to virus clearance.

In an alternative embodiment, the uses and methods of the invention comprise the administration of a protein complex comprising a carrier protein and PC. That protein complex is capable of inducing and/or increasing anti-phosphorylcholine antibodies in the individual.

As will be appreciated, and as described herein, PC is too small by itself to elicit an immune response (for example, in vivo) and thus, PC lacks antigenicity on its own. Such molecules are known generally as haptens. Anti-phosphorylcholine antibodies may therefore only be able to recognise PC when PC is carried by, or conjugated to, an additional molecule.

For that reason, as discussed above, in an embodiment, the invention involves a protein complex comprising a carrier protein and PC, which complex is capable of presenting PC to the immune system of the individual and thereby inducing and/or increasing anti-phosphorylcholine antibodies in the individual. Preferably, the protein complex induces and/or increases the presence and/or amount of anti-phosphorylcholine antibodies in the individual. Those anti-phosphorylcholine antibodies will bind specifically to a complex comprising a virus protein and phosphorylcholine, and thereby elicit an immune response to the virus, optionally leading to virus clearance.

Preferably, the carrier protein is selected from the group comprising: a virus protein, a bacterial protein.

Suitable carrier proteins are discussed further herein, and are known in the art. As will be appreciated, the carrier protein must be capable of binding to phosphorylcholine in a way such that phosphorylcholine is presented to the immune system of the individual and thereby inducing and/or increasing anti-phosphorylcholine antibodies in the individual.

In a further aspect, the invention provides a method for predicting the prognosis of a virus infection in an individual, the method comprising the steps of:
(a) providing a test sample from the individual;
(b) determining the presence and/or amount of anti-phosphorylcholine antibodies in the test sample; and
(c) predicting the prognosis of the virus infection in the individual on the basis of the determination in Step (b).

In another aspect, the invention provides a use of anti-phosphorylcholine antibodies for predicting the prognosis of a virus infection in an individual.

As described herein and shown in the accompanying Examples, the inventors surprisingly discovered that phosphorylcholine associates with virus protein and that the resulting complex (of virus protein and phosphorylcholine) could be specifically recognised by anti-phosphorylcholine antibodies. Furthermore, as the Examples show, the presence and/or amount of anti-phosphorylcholine antibodies in an individual correlate with the outcome and/or severity of the virus infection.

Accordingly, in one aspect, the present invention provides a means for predicting the prognosis of a virus infection in an individual, based of the presence and/or amount of anti-phosphorylcholine antibodies in the individual.

As is well known, viruses are sub-microscopic infectious particles that are only capable of replicating when inside a suitable host cell. Viruses infect all known life forms, including animals, plants and microorganisms (including bacteria and archaea). Typically, virus infection results in rapid replication of the virus within the infected host cell, such that hundreds or thousands or tens-of-thousands of copies of the virus particle are produced. The resulting virus particles are subsequently released (often following death of the host cell) and may then spread to and infect other host cells.

The term “virus” as described herein includes a virus that is: (i) capable of infecting a target cell, optionally wherein the cell is in an individual as defined herein; and (ii) capable of replication in the target cell. As will be appreciated by those skilled in the art, the general steps of viral replication include: (i) attachment and entry of the virus into the host cell; (ii) penetration and uncoating of the virus within the host cell; (iii) replication and translation of viral nucleic acid into viral protein; (iv) assembly of virus particles containing replicated viral nucleic acid and viral protein; (v) release of virus particles from the host cell.

As is well known, virus infections typically result in a reduction or impairment of the health of the infected individual. For example, where the individual is a multicellular organism (such as a plant, or an animal such as a human), virus infection may damage or destroy a substantial number of host cells, thereby reducing or impairing the usual function of cells or tissues in the individual and leading to disease and/or disorder.

By “virus infection in an individual”, we include that the individual contains one or more replicating virus in a cell in that individual, and preferably, that the virus infection has resulted in a reduction or impairment of the health of the infected individual.

In an embodiment, the virus infection is an acute infection. In another embodiment, the virus infection is a chronic infection. In another embodiment, the virus infection is a latent infection.

By “individual”, we include any individual capable of being infected by a virus. In a preferred embodiment, the individual is a human, or an animal (such as a fish, bird, reptile, amphibian or mammal). Mammals include but are not limited to primates (including humans), cows, sheep, goats, horses, dogs, cats, mink, rabbits, guinea pigs, hamsters, ferrets, rats, mice; or bovine, ovine, equine, canine, feline, rodent or murine species. In a preferred embodiment, the subject is a human.

In an embodiment, the individual is male, and preferably a male human (man). Men are generally more susceptible to most viral infections (including influenza viruses, HIV, hepatitis viruses). Moreover, as is discussed in the Examples, a major feature of Covid-19 is that it affects men more severely than women. Accordingly, male individuals, particularly male humans, may particularly benefit from the present invention.

In an alternative embodiment, the individual is female, and preferably a female human (woman). Mean anti-PC antibody levels are generally higher in women than in men. Without wishing to be bound by theory, the inventors therefore believe that—in view of their findings that anti-PC antibodies play a role in viral infection differences in anti-PC antibody levels between males and females may be a contributing factor towards sex-specific differences to viral infections.

In an embodiment, the individual can be selected from: an individual having a virus infection; an individual suspected of having a virus infection; or an individual diagnosed with a virus infection. For example, such an individual could display one or more symptoms of virus infection. Those skilled in the art will be capable of identifying such individuals, which is typically established based on known approaches, such as the identification of clinical symptoms and/or identification in the individual of viral material and/or antibodies against viral material.

It will be appreciated from the present disclosure that the methods and uses of the invention can also be used to predict the prognosis of a virus infection in an individual that has not yet been infected with the virus. Thus, the methods and uses of the invention provide approaches for determining the prognosis of the virus infection if and when that individual is infected at a future point in time. Accordingly, in an alternative embodiment, the individual is a healthy individual (i.e. an individual free from any infection, disease or disorder), and/or is an individual that does not have a virus infection.

In one embodiment, the individual is at risk (for example, high risk) of developing serious viral disease. In another embodiment, the individual is an immunocompromised individual. In other embodiments, the individual is: an elderly adult (for example, a human over 65 years of age); a child younger than two years of age; a healthcare worker; an individual with occupational or recreational contact with animals carrying virus infections (such as birds, pigs and/or bats); a family member in close proximity to a virus infected individual; an individual in contact with individuals with a confirmed or suspected virus infection; or an individual with underlying medical conditions that increase the risk of virus infection and/or serious viral disease (for example, an individual with increased risk of pulmonary infection, heart disease or diabetes).

By “predicting the prognosis of a virus infection”, we include the meaning of predicting how well or badly an individual will be affected by a virus infection. The term prognosis includes the likely or expected outcome or course of the virus infection. Prognoses can include information about symptoms that will develop, improve, remain stable, or worsen; the likelihood of medical or health complications; and/or the likelihood of survival of the individual.

Symptoms of a virus infection can include one or more of the following: fever, cough, shortness of breath, pneumonia, diarrhoea, acute respiratory distress syndrome (ARDS), organ failure (such as kidney failure and renal dysfunction), septic shock, multisystem inflammatory syndrome in children (MIS-C), chronic covid syndrome (CCS), and/or death.

It will be appreciated that it is possible to categorise individuals based on how well or badly that individual is affected by a virus infection. For example:
An individual may have or may develop a “mild” form of the disease. In one embodiment, mild forms of the disease will not require the individual to be hospitalised and/or the individual may only have or develop mild disease symptoms (such as a mild form of one or more of the symptoms listed above). In the accompanying Examples, a “mild” form of disease is referred to as Severity Group 1.
An individual may have or may develop a “moderate” form of the disease. In one embodiment, moderate forms of the disease may cause an individual to be hospitalised and, for example, fitted with a nasal cannula only. In one embodiment, the individual may only have or develop moderate disease symptom (such as a moderate form of one or more of the symptoms listed above). In the accompanying Examples, a “moderate” form of disease is referred to as Severity Group 2.
An individual may have or may develop a “severe” form of the disease. In one embodiment, severe forms of the disease may cause or require an individual to be hospitalised and, for example, requiring oxygen (for example by being fitted with a face mask for oxygen delivery) and/or transferred to an intensive care unit. In one embodiment, the individual may have or develop severe disease symptoms (such as a severe form of one or more of the symptoms listed above, which may result in death of the individual). In the accompanying Examples, a “severe” form of disease is referred to as Severity Group 3.

A positive prognosis can indicate that the individual may be free of infection or may have reduced or no viral titres.

The term “test sample” includes any biological sample from the individual, to be tested in the methods and uses of the invention. It will be appreciated that the test sample may comprise one or more tissue, cell and/or biological fluid taken from (such as isolated from) the individual (e.g., blood; serum; plasma; serum plasma; urine; saliva; intestinal cells; biopsy; stool).

It will also be appreciated that the methods and uses of the invention may be performed using tissues, cells and/or biological fluids when present within an individual. Accordingly, the detection method of the invention can be used to detect a virus infection in a test sample in vitro as well as in vivo. Preferably, the test sample is serum plasma, which has preferably been isolated from the individual.

Phosphorylcholine (PC) is a small molecule composed of a negatively charged phosphate group bonded to a small, positively charged choline group. It is a polar head group of many phospholipids found in cellular membranes and may also exist as a free molecule in multicellular organisms (including humans).

PC is a known danger-associated molecular pattern (DAMP), and an antigen in oxidized low-density lipoprotein (OxLDL), where it becomes exposed on oxidized phospholipids during LDL-oxidation. OxLDL is abundant in atherosclerotic plaques together with dead cells, and OxLDL may be a cause of the inflammation typical of these lesions, activating immune competent cells including monocytes, dendritic cells (DC) and T cells.

PC is also exposed on dead cells (Frostegård. BMC Med. 2013; 11:117). PC is also a pathogen-associated molecular pattern (PAMP) and an antigen on bacteria, parasites and nematodes. PC therefore has a role in eliciting elimination of dead cells by the immune system.

By phosphorylcholine has the formula:

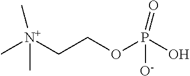

The term phosphorylcholine as used herein also includes phosphorylcholine-associated molecules, such as 2-methacryloyloxyethyl phosphorylcholine.

As explained above and herein, the prognostic methods and uses of the invention are based on the determination of the presence and/or amount of anti-phosphorylcholine (“anti-PC”) antibodies in a test sample.

Anti-PC antibodies are antibodies that are capable of specifically binding to PC. Such anti-PC antibodies are natural antibodies that belong to the innate immune system (Binder et al (2005). J Lipid Res, 2005. 46(7); 1353-63). Natural antibodies have scavenging functions and are a part of the first line defence against certain diseases or disorders. Thus, these antibodies can recognise PC-containing epitopes of certain infectious agents such as some parasites and bacteria and can also recognise PC (neo)epitopes formed in membranes during cell ageing and senescence, and during inflammation.

It has long been known that PC associates with certain parasites and bacteria, and in certain parasites and bacteria, PC is a normal part of the cell wall. However, the binding of PC to virus proteins has not previously been described. This is an unexpected and surprising finding. Additionally, it is surprising that the association between PC and the virus proteins plays a role in the immune response against viruses. This is the first time that PC has been shown to bind to viruses and that this binding has been shown to play a role in the body's defence against viral infections, by PC acting as an antigen.

In biological membranes, these immunogenic PC epitopes are generated by oxidative and/or enzymatic modification of the membrane phospholipid phosphatidylcholine. Membranes containing immunogenic PC induce inflammation in other cells, and that this inflammation can be blocked by anti-PC antibodies (Chang et al (2004). J Exp Med. 200(11); 1359-1370). Further, anti-PC antibodies can inhibit the uptake of oxidized LDL particles by macrophages (Boullier et al (2000). J Biol. Chem, 2000. 275(13); 9163-9169).

Anti-PC antibodies are present in healthy adults, and 5-10% of circulating IgM consists of IgM anti-PC (Frostegård et al., 2013; Rahman et al., 2016; Thiagarajan et al., 2016). Anti-PC antibodies may be polyclonal or monoclonal. Among the most common endogenous anti-PC antibodies found in humans are E01, A01 and D05 (Fiskesund et al (2014). J Immunology. 192(10); 4551-4559).

Low serum levels of anti-PC antibodies have been shown to be related to an increased risk of developing atherosclerosis (WO 2005/100405). Moreover, IgM anti-PC antibodies are associated with protection in several chronic inflammatory disease conditions, including atherosclerosis and cardiovascular disease (CVD), rheumatic diseases and chronic kidney disease (CKD) (Frostegård et al., 2013).

As discussed herein and shown in the accompanying Examples, the present invention is based on the inventors' surprising findings that low levels of anti-PC antibodies correlate with the prognosis of a virus infection in an individual.

Anti-PC antibody levels likely play a role in oxidative stress and increased lipid peroxidation, which can lead to escalation of symptoms, causing severe viral disease.

As discussed above, PC is too small to elicit an immune response (for example, in vivo) and thus, lacks antigenicity on its own. Such molecules are known generally as haptens. Therefore, the anti-phosphorylcholine antibodies may only be able to recognise PC when PC is carried by, or conjugated to, an additional molecule.

The ability of an antibody or antibody fragment to bind to phosphorylcholine and/or a phosphorylcholine conjugate may be determined by any suitable method, which will be known to those skilled in the art. One suitable method is Surface Plasmon Resonance (SPR) analysis, which may be used to measure the binding of the antibody to phosphorylcholine and/or a phosphorylcholine conjugate immobilised (for example via an aminophenyl linker) to a solid surface such as the Biacore SPR biosensor.

By “determining the presence of anti-PC antibodies”, we include the meaning of determining whether or not the test sample contains one or more anti-PC antibodies. Preferably, this comprises exposing phosphorylcholine and/or a phosphorylcholine conjugate to a test sample from an individual and detecting antibodies which have bound to PC or the PC conjugate.

By “determining the amount of anti-PC antibodies”, we include the meaning of quantifying the number of anti-PC antibodies present in the test sample.

The antibodies assessed may be total anti-PC antibodies, or may be particular isotypes of anti-PC antibodies, such as the presence or amount of IgM, IgG, IgA, IgD, IgE antibodies, or a combination of two or more antibody isotypes. Preferably, the presence and/or amount of IgM is determined.

Preferably, the virus is selected from the group comprising: Orthomyxovirus, such as influenza virus, isavirus or thogotovirus; Parvovirus, such as adeno-associated virus (AAV) or a recombinant adeno-associated virus (rAAV); Adenovirus, such as adenovirus; Pneumovirus, such as Respiratory Syncytial Virus (RSV); Herpesvirus, such as Herpes Simplex Virus (HSV); Rhabdovirus, such as Vesicular Stomatitis Virus or Maraba Virus; Retrovirus, such as lentivirus or retrovirus, such as gamma retrovirus; Poxvirus, such as vaccinia virus; Paramyxovirus, such as Measles virus or Newcastle Disease virus; Reovirus, such as rotavirus or reovirus; Picornavirus, such as Rhinovirus, Type I Poliovirus, Coxsackievirus (such as Coxsackievirus A21) or Seneca Valley virus; Flavivirus, such as Dengue virus, Yellow fever virus, West Nile virus or Zika virus; Togavirus, such as Alphavirus; Coronavirus, such as SARS-CoV-1 or SARS-CoV-2.

In a preferred embodiment, the virus is a respiratory virus, which results in a respiratory infection in the individual.

Coronaviruses are enveloped spherical particles, the spike glycoproteins (S protein) of which form a crown-like surface. There are four main sub-groupings of coronaviruses, known as alpha, beta, gamma, and delta. The seven coronaviruses that can infect individuals are: 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV or SARS-CoV-1 (the beta coronavirus that causes severe acute respiratory syndrome, or SARS), SARS-CoV-2 (the novel coronavirus that causes coronavirus disease 2019, or COVID-19). When the virus is coronavirus, the coronavirus may be selected from the group comprising: 229E (alpha coronavirus); NL63 (alpha coronavirus); OC43 (beta coronavirus); HKU1 (beta coronavirus); MERS-CoV (the beta coronavirus that causes Middle East Respiratory Syndrome, or MERS); Sars-CoV or Sars-CoV-1 (the beta coronavirus that causes severe acute respiratory syndrome, or Sars); Sars-CoV-2 (the novel coronavirus that causes coronavirus disease 2019, or COVID-19).

Influenza viruses are spherical or pleomorphic particles, containing linear, negative sense single stranded RNA (ssRNA). There are four types of influenza virus, termed influenza viruses A, B, C, and D. Aquatic birds are the primary source of Influenza A virus (IAV), which is also widespread in various mammals, including humans and pigs. Influenza B virus (IBV) and Influenza C virus (ICV) primarily infect humans, and Influenza D virus (IDV) is found in cattle and pigs. IAV and IBV circulate in humans and cause seasonal epidemics, and ICV causes a mild infection, primarily in children. IDV can infect humans but is not known to cause illness. Influenza A viruses are divided into subtypes based on two proteins on the surface of the virus: hemagglutinin (H) and neuraminidase (N). There are 18 different hemagglutinin subtypes and 11 different neuraminidase subtypes. Current subtypes of influenza A viruses that routinely circulate in people include: A(H1N1) and A(H3N2). Symptomatic infections are mild and limited to the upper respiratory tract, but progression to pneumonia is relatively common. When the virus is influenza virus, the influenza virus is typically selected from the group comprising: Influenza A virus (IAV), Influenza B virus (IBV), Influenza C virus (ICV), Influenza D virus (IDV).

Adenoviruses are non-enveloped (without an outer lipid bilayer) viruses and contain an icosahedral nucleocapsid containing linear double-stranded DNA (dsDNA). In humans, there are seven species of adenoviruses, known as Mastadenoviruses A to G.

Its members infect a variety of vertebrate hosts ranging from fish to humans and are allocated to six genera. Members of genus Mastadenovirus infect mammals, those of Aviadenovirus infect birds, Ichtadenovirus has a single fish adenovirus, and strains of genus Testadenovirus occur in turtles. Members of Atadenovirus occur in squamate reptiles, birds, ruminants, marsupials and tortoises, and those of Siadenovirus infect birds, frog and tortoise. There are currently 88 known human adenoviruses (HAdVs) which are associated with different conditions. The severity of infections ranges from subclinical to lethal. These include respiratory disease, conjunctivitis, gastroenteritis, obesity and adipogenesis. When the virus is adenovirus, the adenovirus is typically selected from the group comprising: human adenoviruses (HAdV) HAdV-A, HAdV-B, HAdV-C, HAdV-D, HAdV-E, HAdV-F and HAdV-G.

Rhinoviruses are non-enveloped viruses and are dodecahedral in structure. They contain positive sense ssRNA. The three species of rhinovirus (A, B, and C) include around 160 recognised types of human rhinovirus that differ according to their surface proteins. Human rhinovirus serotype names are of the form HRV-Xn where X is the rhinovirus species (A, B, or C) and n is an index number. Species A and B have used the same index, while Species C has a separate index. The rhinovirus is the most common viral infectious agent in humans and is the predominant cause of the common cold. When the virus is rhinovirus, the rhinovirus is typically selected from the group comprising: HRV-An, HRV-Bn and HRV-Cn.

Respiratory syncytial virus (RSV) is an enveloped virus and is either spherical or filamentous in shape. It contains negative sense ssRNA in a helical nucleocapsid. Its name comes from the fact that F proteins on its surface cause neighbouring cell membranes to merge, creating large multinucleated syncytia. RSV is divided into two antigenic subtypes, A and B, based on the reactivity of the F and G surface proteins to monoclonal antibodies. To date, 16 RSVA and 22 RSVB strains have been identified. These can cause common colds, bronchiolitis and more serious respiratory illnesses such as pneumonia. When the virus is RSV, the RSV is typically selected from the group comprising: RSVA and RSVB.

Parvoviruses are a family of animal viruses, which contain an icosahedral capsid containing single-stranded DNA (ssDNA). A variety of diseases in animals are caused by parvoviruses. Notably, the canine parvovirus and feline parvovirus cause severe disease in dogs and cats, respectively. In pigs, the porcine parvovirus is a major cause of infertility. Human parvoviruses are generally less severe, the two most notable being parvovirus B19, which causes a variety of illnesses including Erythema infectiosum, fifth disease, and human bocavirus 1, which is a common cause of acute respiratory tract illness.

Herpesviruses are enveloped viruses, and contain dsDNA encased within an icosahedral protein cage. Herpesviruses can cause both latent and lytic infections. More than 130 herpesviruses are known, which can infect mammals, birds, fish, reptiles, amphibians, and molluscs. Among the animal herpesviruses are Pseudorabies Virus, which can cause Aujeszky's disease in pigs; Bovine Herpesvirus 1, which can cause Rhinotracheitis and/or Pustular vulvovaginitis in bovine. Herpesviruses are widespread among humans, and include: Herpes Simplex Viruses 1 and 2 (HSV-1 and HSV-2) which cause orolabial herpes and genital herpes; Varicella Zoster Virus (HHV-3) which can cause chickenpox and shingles; Epstein-Barr Virus (EBV or HHV-4) implicated in several diseases, including mononucleosis and some cancers; human cytomegalovirus (HCMV or HHV-5); Human Herpesvirus 6A and 6B (HHV-6A and HHV-6B); Human Herpesvirus 7 (HHV-7); and Kaposi's sarcoma-associated herpesvirus (KSHV or HHV-8).

Rhabdoviruses have a complex bacilliform or bullet-like shape, are enveloped and contain negative-strand ssRNA. They can infect vertebrates, invertebrates, plants, fungi and protozoans. Diseases associated with Rhabdoviruses include Rabies Encephalitis caused by the Rabies Virus, and flu-like symptoms in humans caused by Vesiculoviruses.

Retroviruses are enveloped viruses, and contain two ssRNA molecules, known as a dimer RNA. The term “retro” in retrovirus refers to the reversal (making DNA from RNA) of the usual direction of transcription. Retroviruses insert copies of their genomes into the genome of a host, which is performed by a viral reverse transcriptase enzyme to produce DNA from its RNA genome. The retroviral DNA is then incorporated into the host's genome, at which point the retroviral DNA is referred to as a provirus. The host cell then transcribes and translates the viral genes along with the cell's own genes, producing the proteins required to assemble new copies of the virus. The family is divided into two basic groups orthoretroviruses and spumaviruses. Retroviruses can cause serious diseases in humans, other mammals, and birds. Human retroviruses include HIV-1 and HIV-2, which can cause AIDS; Human T-lymphotropic virus (HTLV).

Poxviruses are generally enveloped, vary in shape depending upon the species, and contain linear dsDNA. Humans, vertebrates, and arthropods serve as natural hosts. There are currently 83 species in this family, divided among 22 genera, which are divided into two subfamilies. Four genera of poxviruses may infect humans: Orthopoxviruses (e.g., smallpox virus, vaccinia virus, cowpox virus, monkeypox virus, rabbitpox virus); Parapoxviruses (e.g., orfvirus, pseudocowpox virus, bovine papular stomatitis virus); Yatapoxviruses (e.g., tanapox virus, yaba monkey tumor virus); and molluscipoxviruses (e.g., molluscum contagiosum virus).

Reovirus are non-enveloped and have an icosahedral capsid which contains double-stranded RNA (dsRNA). Reoviruses are divided into two subfamilies based on the presence (Spinareoviruses) or absence (Sedoreoviruses) of spike proteins on their surface. Reoviruses have a wide host range, including vertebrates, invertebrates, plants, protists and fungi. Phytoreoviruses and oryzaviruses infect plants. In humans, reoviruses can affect (i) the gastrointestinal system (e.g. Rotavirus) causing severe diarrhoea and intestinal distress) and (ii) the respiratory tract.

Rotaviruses are non-enveloped and have a three-layered icosahedral capsid which contains double-stranded RNA (dsRNA). Rotavirus is very widespread, and almost every child in the world will have been infected with a rotavirus at least once by the age of five. It is the most common cause of diarrhoeal disease among infants and young children. Rotavirus can also infect other animals and is a pathogen of livestock. There are nine species of the genus, referred to as A, B, C, D, F, G, H, I and J. Humans are commonly infected by the species rotavirus A. Within rotavirus A there are different strains, called serotypes. As with influenza virus, a dual classification system is used based on two proteins on the surface of the virus. The glycoprotein VP7 defines the G serotypes and the protease-sensitive protein VP4 defines P serotypes. Rotavirus strains include G1P[8], G2P[4], G3P[8], G4P[8], G9P[8] and G12P[8].

Flaviviruses are enveloped spherical viruses, have an icosahedral nucleocapsid and contain positive-sense ssRNA. Most flaviviruses are transmitted by the bite from an infected arthropod (e.g. a mosquito or tick). In the genus Flavivirus there are 53 defined virus species (e.g. West Nile virus, Dengue virus, Tick-borne encephalitis virus, Yellow fever virus, Zika virus). Some of them are insect-specific flaviviruses (ISFs) (e.g., Cell Fusing Agent virus (CFAV), Palm Creek virus (PCV), and Parramatta River virus (PaRV)).

Togavirus is a genus of RNA viruses. Flaviviruses and the Rubella virus were formerly included in the family Togavirus; however, Alphavirus is now the sole genus in the Togavirus family. Alphaviruses are spherical enveloped viruses with spike proteins on their surface and have an isometric nucleocapsid and contain positive-sense ssRNA.

There are 32 alphaviruses, which can infect various vertebrates (e.g., humans, rodents, fish, birds, and larger mammals such as horses), as well as invertebrates. Many alphaviruses can cause human disease. Infectious arthritis, encephalitis, rashes and fever are the most commonly observed symptoms. Alphaviruses of particular public health concern include Venezuelan equine encephalitis (VEEV), Chikungunya virus (CHIKV), Sindbis virus (SINV), Ross River virus (RRV), Mayaro virus (MAYV), Barmah Forest virus (BFV), and O'nyong'nyong virus (ONNV).

In a preferred embodiment, the anti-phosphorylcholine antibodies bind specifically to a complex comprising a virus protein and phosphorylcholine.

The term “protein” as used herein takes its conventional meaning, namely a plurality of amino acids that are linked together via a peptide bond, to form a polypeptide polymer chain. The term “virus protein” includes proteins which are both a component and a product of the virus, and preferably which are encoded by the viral genome. Preferably, a virus protein is a component of the capsid or envelope of the virus.

The term “bind specifically” refers to the selective recognition of a binding molecule (such as an antibody) for a particular target. Antibodies, such as anti-PC antibodies as described herein, that bind specifically to a target (which can be an epitope) are antibodies which bind to that target with greater affinity, avidity, more readily, and/or with greater duration than to other unrelated targets or molecules. In an embodiment, the extent of binding of the antibody to an unrelated target is less than about 10% such as 9, 8, 7, 6, 5, 4, 3, 2, or 1% of the binding of the antibody to the target as measured, e.g., by any of the techniques described herein. Preferably, the antibody does not bind to an unrelated target.

As will be appreciated, the specificity of an antibody for its target can be determined and/or defined based on affinity measurements. Affinity (K_{D}), expressed by the equilibrium constant for dissociation between antigen and antibody, is a measure of the strength of binding between the epitope and the antigen binding site on the antibody: a smaller K_{D} value indicates that the binding strength between antigen binding molecules is stronger (alternatively, affinity can also be expressed as an affinity constant (K_{A}), which is 1/K_{D}). As will be apparent to those skilled in the art, affinity can be determined by any method known in the art and described herein. Any K_{D} value greater than 1×10⁻⁶ M is generally considered to indicate non-specific binding.

In some embodiments, the anti-phosphorylcholine antibody that binds to the complex has a binding affinity from about −400 kcal/mol to about −200 kcal/mol. The binding affinity may be from about −400, −375, −350, −325, −300, −275, −250 kcal/mol to about −325, −300, −275, −250, −225 or −200 kcal/mol, such as from about −325 to about −275 kcal/mol.

Binding specificity of the binding molecule can be determined experimentally by methods known in the art. Such methods comprise but are not limited to Biophysical Biolayer interferometry (BLI), isothermal titration calorimetry (ITC), Western blots, enzyme-linked immunosorbent assay (ELISA), radioimmunoassay (RIA), electrochemiluminescence (ECL), immunoradiometric assay (IRMA), Enzyme immunoassay (EIA), and surface plasmon resonance (SPR).

In one embodiment, the virus protein is a spike protein of a coronavirus, or a portion or variant thereof.

Spike proteins form large protrusions from the surface of coronaviruses, giving them the appearance of having crowns (hence their name; corona in Latin means crown).

The SARS-CoV-2 Spike (S) protein is homo-trimeric and each spike monomer comprises an outer S1 subunit, which harbours the receptor-binding domain (RBD), and a transmembrane S2 subunit which contains functional elements involved in membrane fusion. Preferably, the portion is the receptor binding domain (RBD), or a portion or variant thereof.

As is well known, the coronavirus S protein mediates viral entry into host cells by first binding to a host receptor through the RBD in the S1 subunit and then fusing the viral and host membranes through the S2 subunit. The RBD is a globular domain situated on the distal surface of the spike protein. Two conformations have been observed in the stabilised trimer. Specifically, in one conformation a single RBD is ACE2-accessible (i.e. “up” conformation) while two are not, and in another conformation all three RBDs are receptor inaccessible (i.e. “down” conformation) (Wrapp et al (2020) Science. 367(6483) 1260-1263; Walls et al (2020) Cell 181(2) 281-292).

By “variant”, we include, for example, allelic variants. Typically, these will vary from the given sequence by only one or two or three, and typically no more than 10 or 20 amino acid residues. Typically, the variants have conservative substitutions. It will be appreciated that any such isolated sequence and naturally occurring variants thereof are encompassed by the present invention. Typically, such variants share at least 90% sequence identity with exemplary sequence, more typically 95%, such as 99% sequence identity. For example, by SARS-CoV-2, we include VUI—202012/01' (Variant Under Investigation, year 2020, month 12, variant 01), which is defined by multiple spike protein mutations (deletion 69-70, deletion 144, N501Y, A570D, D614G, P681H, T716I, S982A, D1118H) present as well as mutations in other genomic regions. The variant may or may not comprises a D614G substitution.

In another embodiment, the virus protein is the hemagglutinin protein of influenza virus, or a portion or variant thereof. The sequence for the hemagglutinin protein of the influenza virus includes Uniprot ID: C3W5S1. Hemagglutinin is an integral, type I membrane glycoprotein involved in virus attachment, envelope fusion and neutralisation. It binds to sialic acid-containing receptors on the cell surface, bringing about the attachment of the virus particle to the cell. This attachment induces virion internalisation either through clathrin-dependent endocytosis or through clathrin- and caveolin-independent pathway. Hemagglutinin plays a major role in the determination of host range restriction and virulence and is responsible for penetration of the virus into the cell cytoplasm by mediating the fusion of the membrane of the endocytosed virus particle with the endosomal membrane. Low pH in endosomes induces an irreversible conformational change in Hemagglutinin 2, releasing the fusion hydrophobic peptide.

In another embodiment, the virus protein is the L3 protein of adenovirus, or a portion or variant thereof. The sequence for the L3 protein of the adenovirus includes Uniprot ID: P03252. The L3 protein is a protease and can come from Human adenovirus C serotype 2 (HAdV-2). L3 cleaves viral precursor proteins (pTP, pIIIa, pVI, pVII, pVIII, and pX) inside newly assembled particles giving rise to mature virions. The protease complexes to its cofactor slides along the viral DNA to specifically locate and cleave the viral precursors. Mature virions have a weakened organisation compared to the unmature virions, thereby facilitating subsequent uncoating. Without maturation, the viral particle would lack infectivity and would be unable to uncoat.

In another embodiment, the virus protein is the genome polyprotein of rhinovirus, or a portion or variant thereof. The sequence for the genome polyprotein of the rhinovirus virus includes Uniprot ID: Q82122. The genome polyprotein can come from Human rhinovirus 16 (HRV16). Human rhinoviruses are composed of a capsid that contains four viral proteins, VP1, VP2, VP3 and VP4. In many rhinoviruses, VP1 contains a hydrophobic pocket which is occupied by a fatty acid-like molecule, or so-called ‘pocket factor’. Antiviral agents have been shown to bind to the hydrophobic pocket in VP1, replacing the pocket factor. The presence of the antiviral compound blocks uncoating of the virus and in some cases inhibits receptor attachment.

In another embodiment, the virus protein is a fusion protein of respiratory syncytial virus (RSV), or a portion or variant thereof. The lipid envelope of RSV comprises transmembrane surface proteins (G, F, SH). The sequence of the precursor protein of the fusion glycoproteins (F1 and F2) of the respiratory syncytial virus (RSV) can be Uniprot ID: O36634. The precursor is cleaved at two sites by a furin-like protease to give rise to the mature F1 and F2 fusion glycoproteins. The F glycoprotein is synthesised as a F0 inactive precursor that is heavily N-glycosylated and processed at two sites by a host furin-like protease probably in the Golgi. The cleavage site between p27 and F1 may occur after endocytosis to yield the mature F1 and F2 proteins. Both cleavages are required for membrane fusion and p27 is released from the processed protein.

Preferably, the binding of the anti-phosphorylcholine antibodies reduce and/or inhibit the binding of the virus to its target cell.

By “target cell” we include the meaning of a target cell, such as an animal cell, such as a mammalian cell, such as a human cell, which is a target for infection by a virus, and whose replication machinery will be used by the virus for replication. Preferably, the target is a human cell. Preferably, the target are cells of the lung, such as epithelial cells of the lung.

In general, viruses bind to their targets via cellular entry receptors expressed on the surface of the target cells. For example, in coronavirus infection, the RBD binds to angiotensin-converting enzyme 2 (ACE2). ACE2 is expressed in a number of tissues, but most abundantly in lung alveolar epithelial cells, kidney cells, heart cells, gastrointestinal tract cells and cell in the testes.

By “reduces the binding”, we include the meaning that the binding of the virus to its target cell is reduced. This can be characterised by a reduction in binding affinity of the virus to the receptor on the target cell.

By “inhibits the binding”, we include the meaning that the binding of the virus to its target cell is reduced to the extent that it entirely or substantially prevented. This can be characterised by a reduction in binding affinity of the virus to the receptor on the target cell, to such an extent that the virus is considered to not bind the receptor or target cell.

As shown in the accompanying Example, anti-PC antibodies bind to an epitope within the RBD of coronavirus which partially overlaps with the ACE2-binding motif, thus blocking ACE2 binding by steric hindrance. In addition, it appears that the anti-PC antibodies will, when bound to the neighbouring RBD in the spike trimer, also confer steric hindrance. As the Examples explain, similar effects are expected for other respiratory viruses, including Adenovirus, Influenza, Rhinovirus, and RSV.

Accordingly, as explained herein, anti-PC antibodies reduce and/or inhibit virus entering cells, thus neutralising it, and preventing and/or treating a disease or condition caused by the virus. Thus, anti-PC antibodies may be understood to be “neutralising”, “blocking” and/or “antagonist” molecules.

Additionally or alternatively, the anti-phosphorylcholine antibodies may elicit an immune response, optionally leading to virus clearance.

By “elicit an immune response”, we include the meaning that the binding of anti-PC antibodies provokes and/or increases an immune reaction in the individual, which immune response is directed against the complex and/or virus.

By “virus clearance”, we include the meaning that the amount of virus is reduced such that it is no longer present in the individual at a detectable level, and thus has been cleared. This can be assessed through methods known in the art such as antigenic tests for viral proteins and/or polymerase chain reaction (PCR) for viral nucleic acid sequences.

As will be appreciated, the process of virus clearance may be similar to the cellular and/or immune processes that occur when PC is found on dead cells in the body. Typically, PC on dead cells is bound by anti-PC antibodies, and the resulting antibody-cell complex will attract phagocytic leukocytes (primarily macrophages) and other recruited cells to the site of cell death. Clearance of the dead cell may occur by phagocytosis and/or efferocytosis.

Most preferably, the anti-phosphorylcholine antibodies inhibit the effects of oxidised and/or proinflammatory phospholipids.

Oxidative stress and increased lipid peroxidation are known to be implicated in different viral diseases, such influenza and covid-19, and may be a contributing cause of pathogenesis, immune dysfunction, apoptosis and inflammation. In general, oxidative stress is caused by an imbalance between pro- and antioxidant mechanisms, which promotes lipid- and DNA-oxidation, damage and interestingly, viral infections are associated with decreased antioxidant defences (Chernyak et al, 2020; Laforge et al, 2020).

Reactive oxygen species (ROS) generated during inflammation as a result of, for example, a virus infection, can cause the oxidation of some phospholipids, such as low-density lipoprotein (LDL) to generate oxidized LDL (oxLDL). LDL is a circulating lipoprotein particle that contains lipids with a PC polar head group. An effect of oxidation of LDL, is that PC containing neo-epitopes that are not present on unmodified LDL are generated. Newly exposed PC on oxLDL act as a danger signal and is recognised by scavenger receptors on macrophages, such as CD36, and the resulting macrophage-engulfed oxLDL proceeds towards the formation of proinflammatory foam cells in the vessel wall. Oxidized LDL is also recognised by receptors on endothelial cell surfaces and has been reported to stimulate a range of responses including endothelial dysfunction, apoptosis, and the unfolded protein response (Gora et al (2010). FASEB 1. 24(9); 3284-3297). PC neo-epitopes are also exposed on LDL following modification with phospholipase A2 or amine reactive disease metabolites, such as aldehydes generated from the oxidation of glycated proteins.

By “inhibiting the effects of oxidised and/or proinflammatory phospholipids”, we include the meaning that anti-PC antibodies can bind to oxidised, or otherwise modified phospholipids and block the pro-inflammatory activity. Without wishing to be bound by theory, the inventors believe that this effect could account for or contribute to low levels of anti-PC antibodies promoting a pro-inflammatory state in the individual and affecting disease severity and outcome. Anti-PC antibodies would lower the levels of oxidised and proinflammatory phospholipids and ameliorate their effects. Thus, a high level of anti-PC antibodies would protect against the pro-inflammatory state of virus infections.

As will be appreciated, inhibiting the effects of oxidised and/or proinflammatory phospholipids may be of particular therapeutic benefit in individuals infected with coronavirus, such as Covid-19. Levels of oxidised phospholipids were found to be elevated in the patients with Covid-19 compared to non-infected individuals (Akpinar et al, 2021. Eur Rev Med Pharmacol Sci. 25:5304-5309).

In some embodiments, the phosphorylcholine binds to the virus protein, or a portion or variant thereof with a binding affinity from about −5.75 kcal/mol to about −2.75 kcal/mol. The binding affinity may be from about −5.75, −5.5, −5.25, −5.0, −4.75, −4.5, −4.25 or −4.0 kcal/mol to about −4.5, −4.25, −4.0, −3.75, −3.5, −3.25, −3.0 or −2.75 kcal/mol, such as from about −4.5 to about −5.5 kcal/mol.

Another way to measure binding affinity is using the inhibition constant (Kᵢ) for the complex between the enzyme (E) and the inhibitor (I) is the dissociation constant for the enzyme inhibitor complex (EI): EI⇄E+I. The smaller the Kᵢ, the greater the binding affinity and the smaller amount of medication needed in order to inhibit the activity of that enzyme.

In another embodiment, the phosphorylcholine binds to the virus protein, or a portion or variant thereof with an inhibition constant (Kᵢ) from about 0 mM to about 5 mM. The inhibition constant may be from about 0, 0.5, 1, 1.5, 2, 2.5, 3 or 3.5 mM to about 1.5, 2, 2.5, 3, 3.5, 4, 4.5 or 5 mM, such as from about 1 to about 4 mM.

Preferably, the binding of the phosphorylcholine to the virus protein permits virus entry into a target cell.

As described above, in order to survive and successfully infect a host, in a first instance, a virus will attach and enter into a target cell. By “permitting entry”, we include the meaning that the binding of PC (alone) to the virus enables (and/or at least does not inhibit) the virus from binding and entering its target cell.

A person skilled in the art would be able to assess the ability of a virus to enter a target cell using methods known in the art. Such methods could include, for example, cell culture assays where viral protein responsible for binding the target cell (such as the spike protein) is stained with a fluorescent marker. The presence of the stained viral protein (such as the spike protein) in the cell could then be compared in the presence of absence or PC. Quantification of these results could be performed using flow cytometry methods. Such flow cytometry methods are well known in the art.

It is preferred that when the method or use is for predicting the prognosis of a virus infection in the individual, the anti-phosphorylcholine antibodies are IgM, IgG and/or IgA anti-phosphorylcholine antibodies.

Different isotypes of immunoglobulin are found in different species. For example in mammals, the different types of immunoglobulins include IgM, IgG, IgA, IgD, IgE. Each isotype also includes different subtypes. For example, subtypes of IgG include IgG1, IgG2, IgG3, IgG4 and subtypes of IgA include IgA1 and IgA2. It will be appreciated that there are different isotypes and subtypes in different species. For example, birds have three immunoglobulin isotypes (IgY, IgM and IgA).

In one embodiment, lower levels of anti-phosphorylcholine antibodies are associated with a worse prognosis.

By “lower levels of anti-PC antibodies”, we include the meaning that levels of IgM anti-PC antibodies in an infected individual predicted to have a worse prognosis are lower when compared to levels of IgM anti-PC antibodies in an infected individual predicted to have a better prognosis.

In an embodiment, the levels of IgM anti-PC antibodies are lower by 10% or 20% or 30% or 40% or 50% or 60% or 70% or 80% or 90% or 100% or more, for example 200% or 300%, compared to the levels in an infected individual predicted to have a better prognosis. In another embodiment, the levels of IgM anti-PC antibodies are lower by 2-fold, or 3-fold, or 4-fold, or 5-fold, or 10-fold or more, compared to the levels in an infected individual predicted to have a better prognosis.

By “worse prognosis”, we include the meaning that the prognosis of an infected individual with lower levels of IgM anti-PC antibodies is worse than the prognosis of an infected individual with higher levels of IgM anti-PC antibodies, for example, in terms of disease severity and/or outcome (including the likelihood of severe symptoms and death).

Additionally or alternatively, lower levels of anti-phosphorylcholine antibodies are associated with more severe disease.

By “more severe disease”, we include the meaning that the disease symptoms of an infected individual with lower levels of IgM anti-PC antibodies are more severe than the disease symptoms of an infected individual with higher levels of IgM anti-PC antibodies.

Additionally or alternatively, lower levels of anti-phosphorylcholine antibodies could be associated with a higher chance of death.

By “higher chance of death”, we include the meaning that chance of death in an infected individual with lower levels of IgM anti-PC antibodies is higher than the chance of death in infected individual with higher levels of IgM anti-PC antibodies.

In an embodiment, the chance of death in the individual with lower levels of IgM anti-PC antibodies is increased by 10% or 20% or 30% or 40% or 50% or 60% or 70% or 80% or 90% or 100% or more, for example 200% or 300%, compared to chance of death in an infected individual with higher levels of IgM anti-PC antibodies. In another embodiment, the chance of death in the individual with lower levels of IgM anti-PC antibodies is increased by 2-fold, or 3-fold, or 4-fold, or 5-fold, or 10-fold or more, compared to the chance of death in an infected individual with higher levels of IgM anti-PC antibodies.

Thus, as shown in the accompanying Examples, the infected individual's level of anti-PC antibodies correlates negatively with the progression of the virus infection. Those who have higher anti-PC antibody levels are more protected from a virus infection, or have a better prognosis, a less severe disease, and/or a higher chance of survival.

Without wishing to be bound by any theory, the inventors believe that at least two mechanisms are involved. As discussed above, firstly anti-PC antibodies are able to reduce and/or inhibit virus entry into the target cells. Additionally, another mechanism involves an increase in elimination of virus by the immune system, as PC acts as a Danger associated molecular pattern (DAMP) and Pattern associated molecular pattern (PAMP).

In one embodiment, determining the presence and/or amount of anti-phosphorylcholine antibodies in the test sample in Step (b) comprises assessing the presence and/or amount of IgM, IgG and/or IgA anti-phosphorylcholine antibodies.

The assessment could be in an individual in whom it is not known whether or not a virus infection has occurred. Alternatively, the assessment could be performed in an individual in whom it is known that a virus infection has occurred. The assessment may be performed, for example at 2, 3, 4, 5, 6, 7, 8, 9, 10, 11, 12 weeks post-infection.

Preferably, step (b) comprises determining the presence and/or amount of anti-phosphorylcholine antibodies using an immunoassay.

Appropriate immunoassays will be well known to those skilled in the art. As is well known, immunoassays can be competitive or non-competitive.

In a typical competitive immunoassay, the anti-PC antibody in the test sample competes with a labelled antibody to bind the PC conjugate. The amount of labelled antibody bound to the PC conjugate is then measured. There is an inverse relationship between concentration of anti-PC antibody in the sample and the quantity of labelled antibody detected.

In non-competitive immunoassays, anti-PC antibody in the sample is bound to the PC conjugate, and then a labelled detection reagent (typically an anti-immunoglobulin antibody) is bound to the anti-PC antibody. The amount of labelled detection reagent bound to the anti-PC antibody is then measured. Unlike the competitive method, the results of the non-competitive method will be directly proportional to the concentration of the anti-PC antibody.

In a non-competitive immunoassay or Western blot, a labelled detection reagent, typically an anti-immunoglobulin antibody, is used to detect anti-PC antibody bound to the PC conjugate. A suitable anti-immunoglobulin antibody must bind specifically to immunoglobulin of the species from which the sample is obtained. It may bind to all immunoglobulin isotypes of that species, or only a subset of isotypes. For example, it may bind only to IgA, IgD, IgE, IgG or IgM, or combinations of two or more of these isotypes. The anti-immunoglobulin antibody may bind specifically only to certain subtypes of any given isotype. Subtypes of human IgA are IgA1 and IgA2. The anti-immunoglobulin antibody may bind to one or both of these subtypes. Subtypes of human IgG are IgG1, IgG2, IgG3 and IgG4. The anti-immunoglobulin antibody may bind to one or more of these human IgG subtypes. It will be appreciated that there are different isotypes and subtypes in different vertebrate species, and those skilled in the art will be able to select and use an appropriate anti-immunoglobulin antibody.

The immunoassay may be performed using radioimmunoassay. In radioimmunoassay, the antibody or detection reagent is labelled with a radioisotope, such as ¹³¹I or ¹²⁵I. In enzyme immunoassays, the antibody or detection reagent is labelled with an enzyme. Suitable enzymes are capable of being detected with the use of a chromogenic substrate. A chromogenic substrate is a substance which, as a result of the reaction with the enzyme, gives rise to a coloured product which can thus be detected spectrophotometrically. Enzymes such as horse radish peroxidase, alkaline phosphatase, beta-galactosidase, and pyrophosphatase from E. coli have been widely employed. Chemiluminescent systems based on enzymes such as luciferase can also be used. Other labels include fluorescent labels such as fluorophores of the Alexa series.

Conjugation of the antibody or detection reagent with the vitamin biotin is frequently used since this can readily be detected by its reaction with enzyme- or fluorophore-linked avidin or streptavidin to which it binds with great specificity and affinity.

In a typical non-competitive enzyme immunoassay, the sample to be analysed is placed in contact and incubated with the PC conjugate adsorbed on a solid substrate. Any anti-PC antibodies present in the sample are specifically bound by the PC conjugate adsorbed on the solid substrate, producing a PC conjugate/anti-PC antibody complex. The sample is then separated from the solid substrate so as to eliminate non-bound materials, for example, by washing. In the next step of the method, an indicator antibody capable of binding any anti-PC antibodies that are present on the substrate in the form of a PC conjugate/anti-PC antibody complex is added to the solid substrate, thus producing a PC conjugate/anti-PC antibody/indicator antibody complex. The indicator antibody may, for example, be an anti-human IgG immunoglobulin raised in a non-human animal species. Finally, the presence of the PC conjugate/anti-PC antibody/indicator antibody complex on the solid substrate is detected, the presence of said complex on the solid substrate being indicative of the presence of anti-PC conjugate antibodies in the sample from the individual.

Typically, the solid substrate is a micro-titration plate, for example, of the type commonly used for performing ELISA immunological assays. The micro-titration plate is preferably a polystyrene plate. Other suitable solid substrates are latex particles, beads and coated red blood cells. Conveniently, the PC conjugate is adsorbed to the solid substrate by incubating the PC conjugate in a buffer with the solid substrate. Suitable buffers include carbonate buffer or phosphate buffered saline. Alternatively, the PC conjugate may be covalently linked to the solid substrate. Typically, after adsorption or covalent linkage of the PC conjugate to the solid substrate, the solid substrate is incubated with a blocking agent to reduce non-specific binding of matter from the sample to the solid substrate. Suitable blocking agents include bovine serum albumin. It is preferred that a quantitative estimate of antibody which can bind to PC, or the PC conjugate is obtained by one or more of the above techniques. In typical non-competitive assays, a linear relationship between the measured variable, whether it be optical density or some other read-out, and antibody concentration, is assumed. For example, if sample A has double the optical density of sample B in the assay (background having been subtracted from both), it is assumed that the concentration of antibody is double in A compared to B. However, it is preferable to construct a standard curve of serial dilutions of a pool of positive serum samples. Preferably, such dilutions are assayed at the same time as the test samples. By doing this, any variation from the linear relationship may be taken into account in determining the quantity of antibody in the samples.

A skilled person would be aware of other methods suitable for determining the presence and/or amount of anti-phosphorylcholine antibodies.

In another embodiment, the step of predicting the prognosis of the virus infection in the individual in Step (c) comprises calculating whether the amount of anti-phosphorylcholine antibodies is below the 25^{th} percentile and classifying these individuals as more likely to have worse prognosis, a more severe disease and/or a higher chance of death.

In a given population of individuals, levels of anti-PC antibodies are likely to vary. The level of anti-PC antibodies determined for any given individual may be categorised as high or low by reference to the range observed in the wider population. For example, a level of such antibodies below a particular percentile value determined with reference to the wider population may be categorised as a low level. Suitably, a low level may correspond to a value below the 25^{th} percentile, or below the 20^{th}, 10^{th} or 5^{th} percentile. A high level may correspond to a value of above the 5^{th}, 10^{th}, 20^{th}, or 25^{th} percentile, for example.

WO 2012/010291 described mean levels of anti-PC IgM levels in a population of about 40-50 U/ml, and median levels of about 84 U/ml. Therefore, values in a sample at or below any one or more of these levels, for example, less than about 84, 80, 75, 70, 68, 65, 60, 55, 50, 45, 40, 35, 30, 25, 20, 19, 18, 17, or less U/ml may be considered as being “low”. Anti-PC IgM levels of, or below, about 25-20 U/ml are typically representative of values below the about the 25^{th} percentile, and values under about 17 U/ml are typically representative of values below about the 10^{th} percentile.

Thus, anti-PC levels, such as IgM anti-PC levels, in a test sample at or below about 50, 40, 30, 25, 20, 19, 18, 17, 16, 15, 14, 13, 12, 11, 10 or less U/ml may be particularly associated with an increased risk of having a worse prognosis, a more severe disease and/or a higher chance of death as a result of virus infection.

The Example below describes mean levels of anti-PC IgM levels in healthy controls at about 120-125 arbitrary units (AU). Arbitrary units are compared to a control serum (for example, serum from an individual that does not have a virus infection), against which anti-PC IgM levels are then tested. Therefore, values in a sample at or below any one or more of these levels, for example, less than about 125, 124, 123, 122, 121, 120, 119, 118, 117, 116, 115, 114, 113, 112, 111, 110, 109, 108, 107, 106, 105, 104, 103, 102, 101, 100 or less AU may be considered as being “low”. Anti-PC IgM levels of, or below, about 100-105 AU are typically representative of values below the about the 25^{th} percentile, and values under about 100 AU are typically representative of values below about the 10^{th} percentile.

Thus, anti-PC levels, such as IgM anti-PC levels, in a test sample at or below about 110, 109, 108, 107, 106, 105, 104, 103, 102, 101, 100, 99 or less AU may be particularly associated with an increased risk of having a worse prognosis, a more severe disease and/or a higher chance of death as a result of virus infection.

In another aspect, the invention provides a method for identifying an individual in need of anti-viral therapy, the method comprising the steps of:
(a) providing a test sample from the individual;
(b) determining the presence and/or amount of anti-phosphorylcholine antibodies in the test sample;
(c) predicting the prognosis of a virus infection in the individual on the basis of the determination in Step (b); and
(d) identifying the individual as one in need of anti-viral therapy on the basis of the determination in Step (c).

By “anti-viral therapy”, we include a therapy for treating and/or preventing a virus infection. In an embodiment, anti-viral therapy comprises anti-viral vaccine.

Examples of anti-viral therapies (which therapies are particularly effective for treating coronavirus infections) include: Sotrovimab, Ritonavir-boosted nirmatrelvir (Paxlovid) (AIIa, Remdesivir (BIIa), Bebtelovimab (CIII), Molnupiravir (CIIa), Gimsilumab, Lenzilumab, Namilumab, Otilimab, Mavrilimumab, Nirmatrelvir Fluvoxamine, or a combination thereof. Other examples of anti-viral therapies (which therapies are particularly effective for treating influenza infections) include: Oseltamivir, Baloxavir, Zanamivir, Peramivir, or a combination thereof. Other examples of anti-viral therapies (which therapies are particularly effective for treating adenovirus infections) may include: Cidofovir, Ribavirin, Ganciclovir, Vidarabine, or a combination thereof. Other examples of anti-viral therapies (which therapies are particularly effective for treating Respiratory Syncytial Virus (RSV) infections) include: Palivizumab, Benzimidazole derivatives, Motavizumab, Ribavirin.

Other treatments which can be used to treat Covid-19 and other virus infections may include Corticosteroids, Chloroquine and Hydroxychloroquine. They may also include biologics such as TNF-inhibitors (e.g. Etanercept (Enbrel, and biosimilar Benepali); Infliximab (Remicade, and biosimilars such as Remsima or Inflectra); Adalimumab (Humira and biosimilar as Imraldi); Golimumab (Simponi); Certolizumabpegol (Cimzia)), IL-1-receptor antagonist (e.g. Anakinra (Kineret)), anti-CD-20 antibody (e.g. Rituximab (Mabthera, and biosimilar as Rixathon)), CTLA4 (e.g. Abatacept (Orencia)), IL-6-receptor antagonist (e.g. Tocilizumab (RoActemra) and Sarilumab (Kevzara)), Tofacitinib, Baricitinib.

By an individual in need of anti-viral therapy, we include the meaning of individuals who would benefit from anti-viral therapy. For example, where an individual has a virus infection, such anti-viral therapy would improve the health (for example, by reducing symptoms of the virus infection) and/or reduce the severity of the virus infection and/or improve the likely outcome of the virus infection in the individual. In another embodiment, where the individual has not yet been infected with the virus, such anti-viral therapy would reduce the chance of the individual being infected with the virus and/or reduce the severity of the virus infection when infected.

Preferably, the method for identifying an individual in need of anti-viral therapy further comprises the step of administering an anti-viral therapy to the individual.

Various delivery systems are known and can be used to administer an anti-viral therapy to the individual, e.g., encapsulation in liposomes, microparticles, microcapsules, receptor mediated. Methods of administration include, but are not limited to, intradermal, transdermal, intramuscular, intraperitoneal, intravenous, subcutaneous, intranasal, epidural and oral routes. The anti-viral therapy may be administered by any convenient route, for example by infusion or bolus injection, by absorption through epithelial or mucocutaneous linings (e.g., oral mucosa, rectal and intestinal mucosa, etc.) and may be administered together with other biologically active agents. The anti-viral can be also delivered in a vesicle, in particular a liposome (see, for example, Langer (1990). Science. 249; 1527-1533). Administration can be systemic or local. Those skilled in the art would be capable of selecting an appropriate route of administration, for the particular individual and anti-viral therapy.

In a further aspect, the invention provides an isolated and/or purified protein complex, comprising a carrier protein and phosphorylcholine.

By an “isolated and/or purified” protein complex, we include the meaning of one which is separated from other components which are present in the natural source of the protein complex. An “isolated and/or purified” protein complex, such as a virus protein and PC, can be substantially free of other cellular material, or culture medium when produced by recombinant techniques, or substantially free of chemical precursors or other chemicals when chemically synthesized.

Due to their small sizes, haptens can be “carried” or conjugated to “carrier proteins” to ensure an immune response is elicited. Phosphorylcholine can be linked to a carrier, preferably via a spacer.

Any suitable spacer may be used. Non-limiting examples of spacers include coupling agents (typically, bi-functional compounds), such as a di-carboxylic acids like succinic and glutaric acid, the corresponding di-aldehydes, di-amines such as 1,6 diaminohexane, di-substituted phenols such as p-amino-phenol, p-diazo-phenol, p-phenylenediamine, p-benzoquinone, and the like.

Phosphorylcholine can be covalently or non-covalently linked to the carrier protein. Examples of covalent links may or may not include links formed by cross linking of cysteine or lysine. Examples of non-covalent links may or may not include links formed by electromagnetic interactions such as electrostatic interactions (e.g. ionic, hydrogen, halogen bonding), van der Waals forces, n-effects and hydrophobic interactions. Preferably, PC is linked to the carrier protein via the phosphate group.

In yet another aspect, the invention provides an isolated and/or purified protein complex, comprising a carrier protein and phosphorylcholine, for use in medicine.

The protein complex may be useful in medicine, as the administration of the complex can modulate the presence and/or amount of anti-PC antibodies, which in turn can have a positive effect on the progression of the virus infection or disease. Thus, active immunisation may be used to increase the titre of anti-PC antibodies to a level that when assessed, would not be said to be “low”. In one embodiment, administration of the complex may be used to increase anti-PC antibody levels, which can prevent and/or treat the virus infection.

It is also contemplated herein to use an isolated and/or purified protein complex, comprising a carrier protein and phosphorylcholine prophylactically for subjects at risk of virus infection and developing serious symptoms.

Preferably, the protein complex comprising a carrier protein and phosphorylcholine is administered by injection. However, in practice it can be administered by any suitable means that allows the complex to elicit an immune response in the individual to which it is administered.

In an embodiment, the protein complex is provided in a pharmaceutical composition—that is the protein complex is provided in combination with a pharmaceutically acceptable carrier, excipient or diluent.

The pharmaceutical composition in accordance with the invention may be administered with suitable pharmaceutically acceptable carriers, excipients, and other agents that are incorporated into formulations to provide improved transfer, delivery, tolerance, and the like.

By “pharmaceutically acceptable” we include that the formulation is sterile and pyrogen free. Suitable pharmaceutically acceptable carriers, excipients or diluents are well known in the art of pharmacy. The pharmaceutically acceptable carriers, excipients or diluents must be “acceptable” in the sense of being compatible with the agent of the invention and not deleterious to the recipients thereof. Typically, the pharmaceutically acceptable carriers, excipients or diluents will be water or saline which will be sterile and pyrogen free; however, other pharmaceutically acceptable carriers, excipients or diluents may be used. Appropriate pharmaceutically acceptable carrier, excipient or diluent materials that may be employed in compositions of the invention include relevant materials that, in the appropriate combination, are suitable (and/or approved) for pharmaceutical use and/or delivery, and are capable of maintaining their physical and/or chemical integrity, and/or do not affect the physical and/or chemical integrity of any active ingredients and/or any other ingredients that are or may be present in the composition under normal storage conditions

By “pharmaceutically acceptable carriers”, we also include excipients or stabilisers that are non-toxic to the cell or mammal being exposed thereto at the dosages and concentrations employed. Often the pharmaceutically acceptable carrier is an aqueous pH buffered solution. Examples of pharmaceutically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or non-ionic surfactants such as TWEEN™, polyethylene glycol (PEG), and PLURONICS™ or polyethylene glycol (PEG).

By “diluent” we include the meaning of one which is pharmaceutically acceptable (i.e. safe and non-toxic for administration to an individual, such as a human) and is useful for the preparation of a liquid formulation, such as a formulation reconstituted after lyophilisation. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

In another aspect, the invention provides a method for eliciting anti-phosphorylcholine antibodies in an individual, the method comprising administering to an individual a protein complex as described herein.

In yet another aspect, the invention provides a use of a protein complex comprising a carrier protein and phosphorylcholine as described herein, for eliciting anti-phosphorylcholine antibodies in an individual.

In a further aspect, the invention provides a protein complex comprising a carrier protein and phosphorylcholine as described herein, for use in eliciting anti-phosphorylcholine antibodies in an individual.

In yet a further aspect, the use of a protein complex comprising a carrier protein and phosphorylcholine as described herein in the manufacture of a medicament for eliciting anti-phosphorylcholine antibodies in an individual. Methods of manufacturing a medicament using an active agent, such as the agent of the invention, are well known to persons skilled in the art of medicine and pharmacy.

By “treating a virus infection in an individual”, we include the meaning of reducing or ameliorating the severity of at least one symptom or indication of the virus infection due to the administration of a therapeutic agent such as the protein complex of the present invention to a subject in need thereof. The term includes inhibition of progression of disease or of worsening of infection. The term also includes positive prognosis of disease, i.e., the subject may be free of infection or may have reduced or no viral titres upon administration of a therapeutic agent such as the protein complex of the present invention. The therapeutic agent may be administered at a therapeutic dose to the subject.

By “preventing a virus infection in an individual”, we include the meaning of inhibiting the manifestation of a virus infection and/or any symptoms or indications of a virus infection upon administration of the protein complex of the present invention. The term includes prevention of spread of infection in a subject exposed to a virus or at risk of having a virus infection.

By “effective amount” we include an amount of the protein complex of the invention that is sufficient to treat and/or prevent a virus infection in an individual. An effective amount could be determined in vivo and/or clinical trials

By “pharmaceutical composition thereof”, we include the meaning of a pharmaceutical composition comprising the anti-phosphorylcholine antibody which binds specifically to a complex comprising a virus protein and phosphorylcholine, and a pharmaceutically acceptable carrier, excipient or diluent.

In yet another aspect, the invention provides a protein complex according to the aspect above, or a pharmaceutical composition according to the aspect above, or an anti-phosphorylcholine antibody which binds specifically to a complex comprising a virus protein and phosphorylcholine, or a pharmaceutical composition thereof for use in treating and/or preventing a virus infection in an individual.

In a further aspect, the invention provides a use of a protein complex according to the aspect above, or a pharmaceutical composition according to the aspect above, or an anti-phosphorylcholine antibody which binds specifically to a complex comprising a virus protein and phosphorylcholine, or a pharmaceutical composition thereof in the manufacture of a medicament for use in treating and/or preventing a virus infection in an individual.

A clinician can determine the most appropriate administrative regimen for an individual based on factors such as the individual's weight, age, gender, diagnosis or prognosis, and the half-life of the administered therapeutic molecule. However, in general it may be suitable to treat an individual with a single dose, or multiple doses, of an effective amount of a protein complex according to the aspect above, or a pharmaceutical composition according to the aspect above, an anti-phosphorylcholine antibody which binds specifically to a complex comprising a virus protein and phosphorylcholine or a pharmaceutical composition thereof. Where multiple administrations are made, these may be made at a rate of, for example, once, twice, three times, four times or more often per day, week or month, and may be continued for a period of time necessary and effective to increase the levels of anti-PC antibodies in the individual and thereby obtain a therapeutically or prophylactically beneficial effect in respect of virus infections. For example, treatment may continue for 1, 2, 3, 4, 5, 6, 7, 8, 9, 10, 11, 12 or more days, weeks, months or years, or even for the rest of the life of the subject. In the case of the use of anti-PC antibodies, then administration would most typically be made weekly, or once or twice per month, and continue for as long as is clinically beneficial. In the case of the administration with a protein complex, in one embodiment, the treatment may involve an initial immunisation, followed by a further administration as a booster (for example, within about one month of the initial immunisation), and optionally followed by yearly further administrations, continued for as long as is clinically beneficial. The invention provides active (where the composition comprises at least one PC-conjugate) or passive (where the composition comprises the defined antibody) immunisation having immunogenic or therapeutic properties against virus infections.

One embodiment of the present invention is to use a protein complex, comprising a carrier protein and phosphorylcholine for the preparation of a pharmaceutical composition to be used in the treatment and/or prevention of a virus infection. The complex can, for example, be PC linked to a pharmaceutically acceptable carrier such as a protein, carbohydrate, or polymer. The pharmaceutical composition is preferably given by injection but can in practice be administered by any suitable means that allows the PC-conjugate to provoke an immune response in the individual to which it is administered.

For the purposes of active immunisation of a patient, one or more molecule of the protein complex described herein are prepared in an immunogenic formulation, optionally containing suitable adjuvants and carriers, and administered to the individual in known ways. Suitable adjuvants include Freund's complete or incomplete adjuvant, muramyl dipeptide, the “Iscoms” of EP109942, EP 180564 and EP 231039, alum, aluminium hydroxide, saponin, DEAE-dextran, neutral oils (such as miglyol), vegetable oils (such as arachis oil), liposomes, Pluronic polyols or the Ribi adjuvant system. “Pluronic” is a Registered Trade Mark.

As discussed above, one embodiment of the invention involves the use or administration of anti-PC antibodies for treating and/or preventing a virus infection in an individual. Approaches in which antibodies are administered to an individual in need thereof are generally known, and typically referred to as “passive immunisation” approaches.

Such antibodies may be monoclonal antibodies and can be produced using methods known in the art. Anti-PC monoclonal antibodies can be produced using any standard method known in the art (Briles et al (1982). J Exp Med. 156; 1177-1185; Spira et al (1988). J. Immunology. 140; 2675-2680). Other polyclonal or chimeric antibody preparations may be used, such as anti-PC antibody-enriched preparations obtained from intravenous immunoglobulin preparations. Intravenous immunoglobulin preparations are highly purified preparations of IgG commercially available and are used in the treatment of patients who have no, or very low levels of antibody production.

Additionally, the present invention contemplates the use of recombinantly produced anti-PC antibodies and/or other artificially created anti-PC antibody derivatives, such as CDR-grafted and/or humanised antibodies, scFv, dAb, Fab, F(ab′)2, Fv or other molecules which comprise or consists of PC-binding fragments of an antibody. The antibodies may be human antibodies in the sense that they have the amino acid sequence of human antibodies with specificity for the phosphorylcholine, but they may be prepared using methods known in the art that do not require immunisation of humans. For example, transgenic mice are available which contain, in essence, human immunoglobulin genes (Vaughan et al (1998) Nature Biotechnology. 16; 535-539).

In a preferred embodiment, the use or administration of anti-PC antibodies for treating and/or preventing a virus infection in an individual comprises the use of administration of the E01 and/or A01 and/or D05 anti-PC antibodies (described in Fiskesund et al. 2014), and which are also set out below.

### E01 Antibody:

SEQ ID NO: 1E01-VH (Variable domain Heavy Chain)-Nucleotide Sequence:
GAGGTGCAGCTGGTGGAGTCCGGGGGAGGCTTAATTCAGCCTGGGGGGTCCCTGAGACTC
TCCTGTGCAGCCTCTGGATTCACCTTCAGTAACTACTGGATGCACTGGGTCCGCCAGGCTCC
AGGGAAGGGGCTGGTGTGGGTCTCACGTGTTAATAGTGACGGGACTTCCACAACCTACGCG
GACTCCGTGAAGGGCCGATTCACCATCTCCAGAGACGACGCCAAGAACACGCTGTATCTGC
AAATGCACAGTCTGAGAGCCGAGGACACGGCTGTGTATTACTGTGTGGCAACTCGCCCAGA
TAATGACTACTGGGGCCAGGGAACCCTGGTCACCGTCTCCTCA
SEQ ID NO: 2 E01-VH (Variable Heavy Chain)-Protein Sequence:
EVQLVESGGGLIQPGGSLRLSCAASGFTFSNYWMHWVRQAPGKGLVWVSRVNSDGTSTTYAD
SVKGRFTISRDDAKNTLYLQMHSLRAEDTAVYYCVATRPDNDYWGQGTLVTVSS
SEQ ID NO: 3 E01-VL (Variable domain Light Chain)-Nucleotide Sequence:
TCAGAAATTGTGTTGACGCAGTCTCCAGGCACTCTGTCTTTGCCTCCAGGGGAAAGAGCCAC
CCTGTCCTGCAGGGCCAGTCAGAGTGTTAGCAGCAGCTCCTTAGCCTGGTACCGGCAGAAA
CCTGGCCAGGCTCCCAGACTCCTCATCTATGAGACATCCAGCAGGGCCACTGGCATCCCAGA
CAGGTTCAGTGGCAGTGGGTCTGGGACAGACTTCACTCTCACCATCAGCAGACTGGAGCCT
GAAGATTTTGCAGTGTTTTACTGTCTGCATTATGGTAGCTCATCTTGGACGTTCGGCCAAGG
GACCAAGGTGGAAATCAAA
SEQ ID NO: 4 E01-VL (Variable domain Light Chain)-Protein Sequence:
SEIVLTQSPGTLSLPPGERATLSCRASQSVSSSSLAWYRQKPGQAPRLLIYETSSRATGIPDRFS
GSGSGTDFTLTISRLEPEDFAVFYCLHYGSSSWTFGQGTKVEIK

### A01 Antibody:

SEQ ID NO: 5 A01-VH (Variable domains Heavy Chain)-Nucleotide Sequence:
CAGGTGCAGCTGCAGGAGTCGGGCCCAGGACTGGTGAAGCCTTCGGAGACCCTGTCCCTCA
CCTGCACTGTCTCTGGTGACTCCATCAGTAGTGACTACTGGAGTTGGGTCCGGCAGCCCCCA
GGGAGGGGACTGGAGTGGATTGGTTACGTCTATTATAGTGGGGTCACCAGGTACAATCCCT
CGCTCAACAGTCGAGTCACCATGTTAATAGACACGTCCAAGAAATATTTCTCCCTGAAGTTGA
GGTCTGTGACGGCCGCAGACACGGCCGTGTATTACTGTGCGAGACATTATGAGTCCTCCTAC
GTTAATGGTGGAGGTCAGCAGCAGTCATGGCCAAACTGGTTCGACGCCTGGGGCCACGGAA
TTCTGGTCACCGTCTCCTCA
SEQ ID NO: 6 A01-VH (variable domains) Heavy Chain-Protein Sequence:
QVQLQESGPGLVKPSETLSLTCTVSGDSISSDYWSWVRQPPGRGLEWIGYVYYSGVTRYNPSL
NSRVTMLIDTSKKYFSLKLRSVTAADTAVYYCARHYESSYVNGGGQQQSWPNWFDAWGHGIL
VTVSS
SEQ ID NO: 7 A01-VL (Variable domain Light Chain)-Nucleotide Sequence:
GACATCGTGATGACCCAGTCTCCAGACTCCCTGGCTGTGTCTCTGGGCGAGAGGGCCACCA
TCAACTGCAAGTCCAGCCAGGATATTTTATACAGTCCCAACAAAAAGAATTACTTAGCTTGGT
ACCAGCAGAAACCAGGGCGGCCTCCTAAGATGCTGATTTACTGGGCATCTACCCGGGAATC
CGGGGTCCCTGACCGATTCACCGGCAGCGGGTCTGCGACAGACTTCACTCTCACCATCACCA
GCCTGCAGGCTGAGGATGTGGCAGTTTATTATTGTCAGCAGTCATATTCTACGCCGTTCACTT
TCGGCCCTGGGACCAAAGTGGATATCAAACGT
SEQ ID NO: 8 A01-VL (Variable domain Light Chain)-Protein Sequence:
DIVMTQSPDSLAVSLGERATINCKSSQDILYSPNKKNYLAWYQQKPGRPPKMLIYWASTRESGV
PDRFTGSGSATDFTLTITSLQAEDVAVYYCQQSYSTPFTFGPGTKVDIKR

### D05 Antibody:

SEQ ID NO: 9 D05-VH (Variable domain Heavy Chain-Nucleotide Sequence:
GAGGTGCAGCTGTTGGAGTCTGGGGGAGGCTTGGTACAGCCGGGGGGGTCCCTGAGACTC
TCCTGTGCAGCCTCTGGATTCACCTTTAGCAATTATGCCATGACCTGGGTCCGCCAGGCTCC
AGGGAAGGGGCTGGAGTGGGTCTCAAGTATGAGTGCTAGTGATGGTGGCACATACTATGCA
GACTCCGTGAAGGGTCGGTTCATTATTTCGAGAGACAATTCCAACGGCGCGCTGTTTCTGCA
AATGAACAGCGTGAGAGTCGAGGACACGGCCATGTATTTCTGTACGAAAGAAATTTGGCATA
ATAGCGATTACGGTAACCCCCGGGAAGAGAGCTTCTGGGGCCAGGGAACCCTGGTCACCGT
CTCCTCA
SEQ ID NO: 10 D05-VH (Variable domain Heavy Chain)-Protein Sequence:
EVQLLESGGGLVQPGGSLRLSCAASGFTFSNYAMTWVRQAPGKGLEWVSSMSASDGGTYYAD
SVKGRFIISRDNSNGALFLQMNSVRVEDTAMYFCTKEIWHNSDYGNPREESFWGQGTLVTVSS
SEQ ID NO: 11 D05-VL (Variable domain Light Chain)-Nucleotide Sequence:
GACATCCAGATGACCCAGTCTCCCTCCTCACTGTCTGCGTCTGTAGGAGACAGAGTCGCCAT
CACTTGTCGGGCGAGTCAGGACATTAAAAATTACGTAGCCTGGTTTCAGCAGAAACCAGGGA
AAGCCCCTAAGTCCCTGATCTTTGCTGCATCCAGTTTGCAAGGTGGGGTCCCATCAAAGTTC
AGCGGCAGTGCGTCTGGGACAGATTTCACTCTCACCATCAGCAGCCTGCAGCCTGAAGATTT
TGCAACTTATTACTGCCAACAATATTATAGTTACCCTCTCGCTTTCGGCGGGGGGACCAAGGT
GGAGATCAAA
SEQ ID NO: 12 D05-VL (Variable domain Light Chain)-Protein Sequence:
DIQMTQSPSSLSASVGDRVAITCRASQDIKNYVAWFQQKPGKAPKSLIFAASSLQGGVPSKFS
GSASGTDFTLTISSLQPEDFATYYCQQYYSYPLAFGGGTKVEIK

Such antibodies could be given to an individual as a treatment for an ongoing viral disease, one example being Covid-19, in order to ameliorate the symptoms.

The proposed methods active and passive immunisation will modulate (preferably, increase) the titre of anti-PC antibodies which in turn will have a positive effect on the development of virus infections (that is, the development of virus infections will be reduced). Thus, active and passive immunisation may be used to increase the titre of anti-PC antibodies to a level that, when assessed by the methods of diagnosis according to the present application, would not be said to be “low” or indicative of an increased risk of development, or progression of severe viral infections.

Thus, active and passive immunisation according to the present invention may be used to increase anti-PC antibody levels, such as IgM anti-PC levels, in an individual to a level that is greater than about 10, 15, 20, 25, 30, 35, 40, 45, 50, 55, 60 or 65 U/ml or to a level that is greater than about 99, 100, 103, 106, 109, 112, 115, 118, 121, 122 AU. Accordingly, the method of active or passive immunisation according to the present invention may be used to increase anti-PC antibody levels to a level that is above the mean and/or median average, or above a particular percentile value determined with reference to the wider population, such as above the 5^{th}, 10^{th}, 20^{th} or 25^{th} percentile.

In another embodiment, active and/or passive immunisation according to the present invention may be used to increase anti-PC antibody levels in an individual who is not considered to have low levels of anti-PC antibodies, in order to increase anti-PC antibody levels. Thus, active and/or passive immunisation may increase anti-PC antibody levels (such as anti-PC IgM antibody levels) by 10% or 20% or 30% or 40% or 50% or 60% or 70% or 80% or 90% or 100% or more, for example 200% or 300%, compared to the levels usually found in the individual. In another embodiment, active and/or passive immunisation may increase anti-PC antibody levels (such as anti-PC IgM antibody levels) by 2-fold, or 3-fold, or 4-fold, or 5-fold, or 10-fold or more, compared to the levels usually found in the individual.

Preferably the carrier protein is selected from the group comprising: a virus protein, a bacterial protein. Suitable carrier proteins are discussed further herein, and are known in the art. As will be appreciated, the carrier protein must be capable of binding to phosphorylcholine in a way that presents phosphorylcholine to the immune system of the individual and thereby induces and/or increases anti-phosphorylcholine antibodies in the individual.

By being “presented”, we include the meaning that the carrier protein interacts with PC, in such a way that some or all of PC is accessible to other binding molecules. Preferably, the PC is presented or exposed in such a way that it is not buried within the carrier protein. In this way, PC is presented to the immune system, and in turn anti-PC antibodies are raised against this antigen.

The carrier protein can be, for example, a protein-PC conjugate, such as a human serum albumin (HSA)-PC conjugate, a transferrin-PC conjugate, a keyhole limpet hemocyanin (KLH)-PC conjugate or a bovine serum albumin (BSA)-PC conjugate, a virus protein (e.g. the coronavirus spike protein).

Preferably, the virus from the aspects above is selected from a group comprising: Orthomyxovirus, such as influenza virus, isavirus or thogotovirus; Parvovirus, such as adeno-associated virus (AAV) or a recombinant adeno-associated virus (rAAV)); Adenovirus, such as adenovirus; Pneumovirus, such as Respiratory Syncytial Virus (RSV); Herpesvirus, such as Herpes Simplex Virus (HSV); Rhabdovirus, such as Vesicular Stomatitis Virus or Maraba Virus; Retrovirus, such as lentivirus or retrovirus, such as gamma retrovirus; Poxvirus, such as vaccinia virus; Paramyxovirus, such as Measles virus or Newcastle Disease virus; Reovirus, such as rotavirus or reovirus; Picornavirus, such as Rhinovirus, Type I Poliovirus, Coxsackievirus (such as Coxsackievirus A21) or Seneca Valley virus; Flavivirus, such as Dengue virus, Yellow fever virus, West Nile virus or Zika virus; Togavirus, such as Alphavirus; Coronavirus, such as SARS-CoV-1 or SARS-CoV-2.

In a further aspect, the invention provides a method, use, protein complex or pharmaceutical composition substantially as described herein with reference to the accompanying description, examples, claims and/or figures.

The listing or discussion in this specification of an apparently prior-published document should not necessarily be taken as an acknowledgement that the document is part of the state of the art or is common general knowledge.

Preferred, non-limiting examples which embody certain aspects of the invention will now be described, with reference to the following figures:

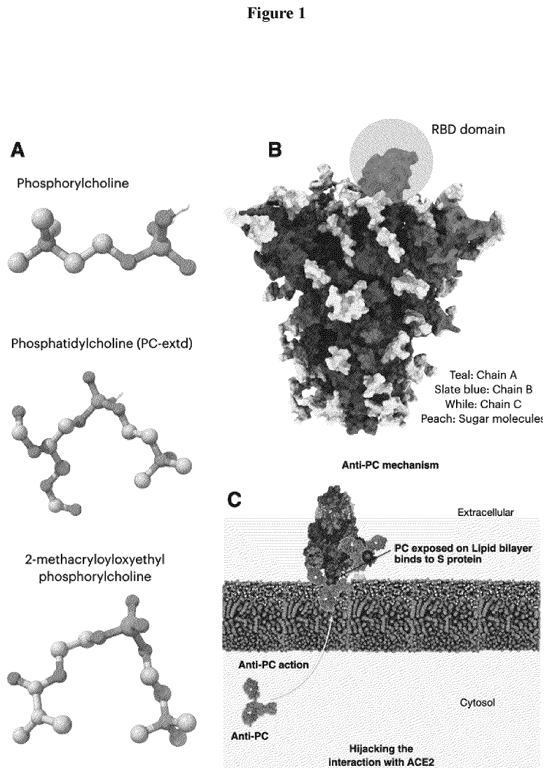

FIG. 1: A: Schematic representation of the molecules taken in this study to understand the interaction mechanism with (B) SARS-CoV-2 Spike protein. C: Mechanistic illustration of the anti-PC action against SARS-CoV-2.

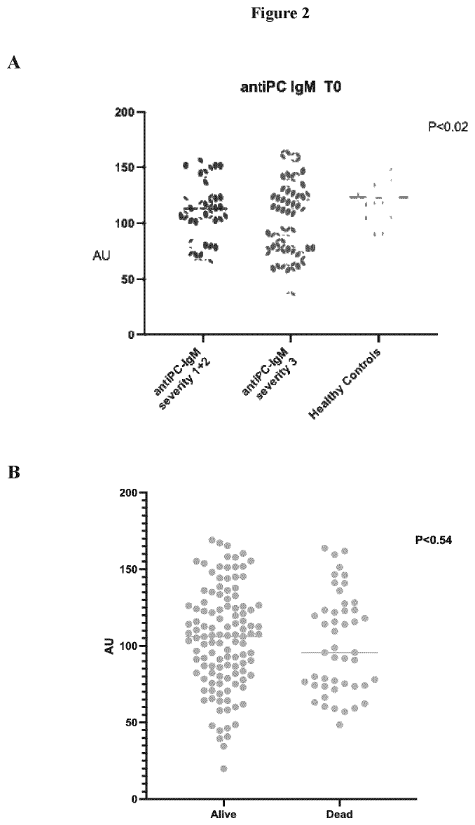

FIG. 2: A: IgM anti-PC levels among the most severely sick COVID-19 patients as compared to less severely sick, and healthy controls. B: Mortality in relation to IgM anti-PC levels at baseline.

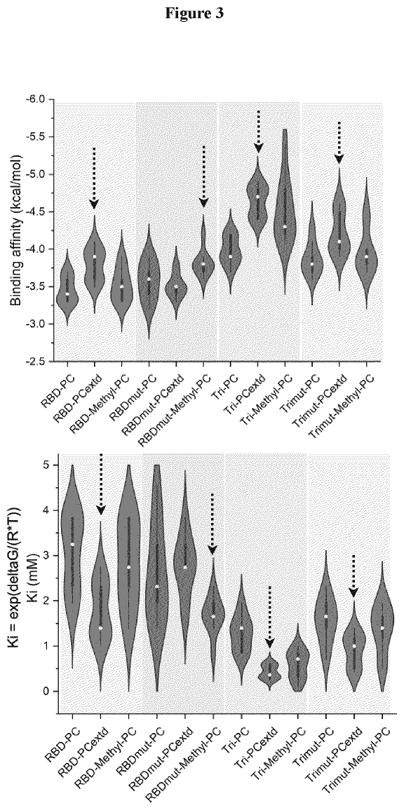

FIG. 3: Violon plots depicting the binding affinities in kcal/mol and Inhibition constant Ki (mM) for all the molecules against wildtype and mutant form.

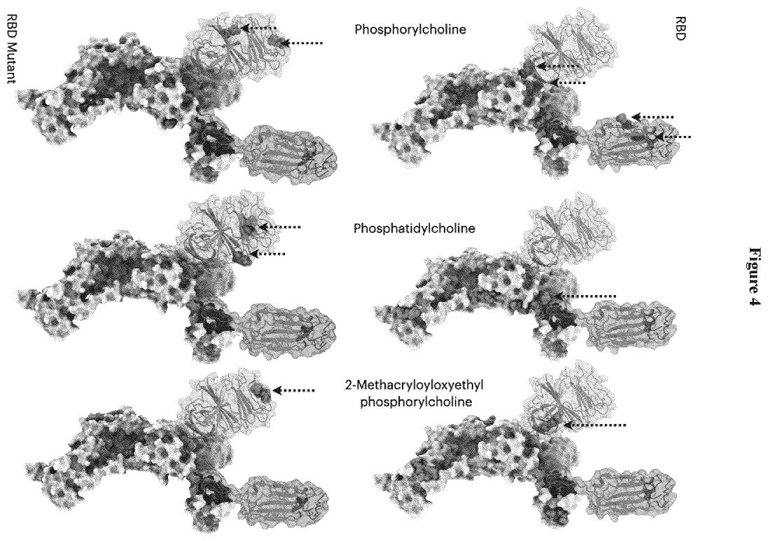

FIG. 4: Structural representation of the binding of PC associated molecules to RBD wildtype and mutant form. The arrow indicates the PC binding sites.

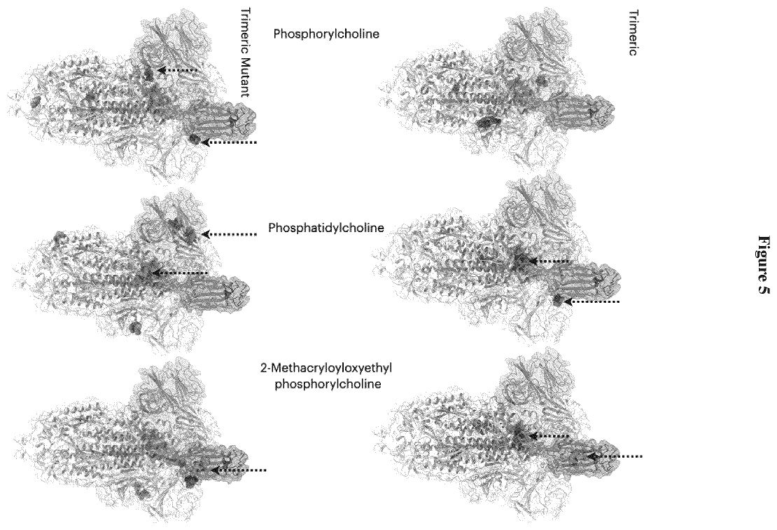

FIG. 5: Structural representation of the binding of PC associated molecules to trimeric spike wildtype and mutant form. The arrow indicates the PC binding sites.

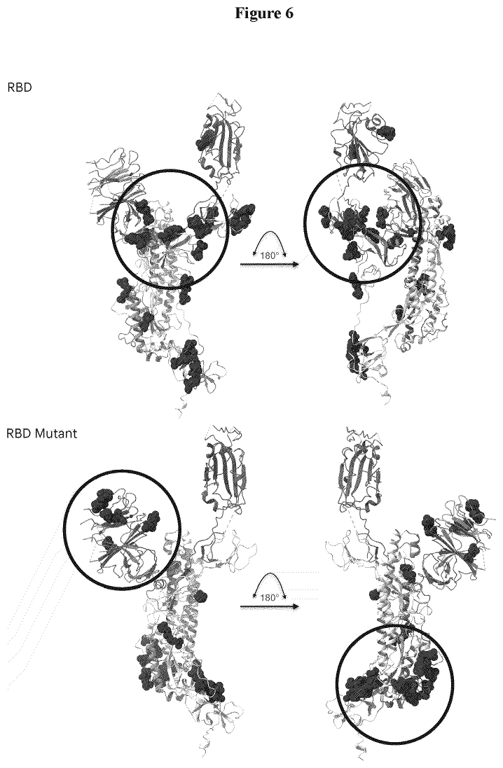

FIG. 6: Overall Structural representation of the binding of PC associated molecules to RBD wildtype and mutant form. The circle represents the high affinity molecules.

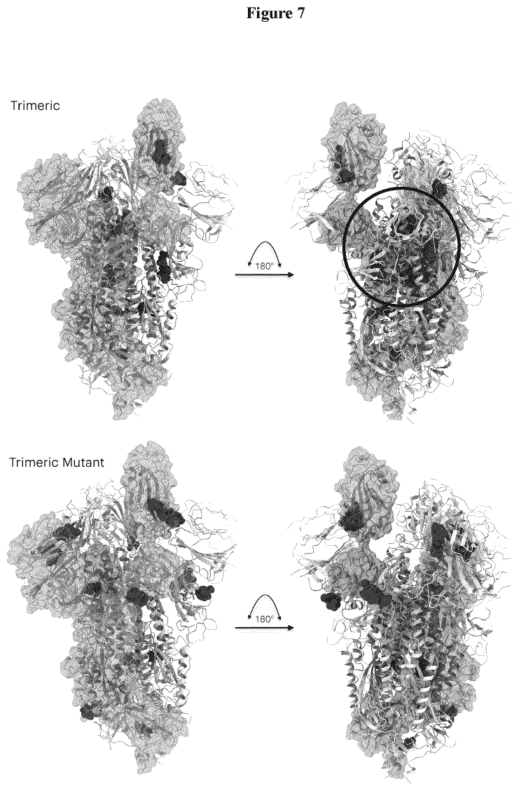

FIG. 7: Overall Structural representation of the binding of PC associated molecules to trimeric spike wildtype and mutant form. The circle represents the high affinity binding conformations of PC.

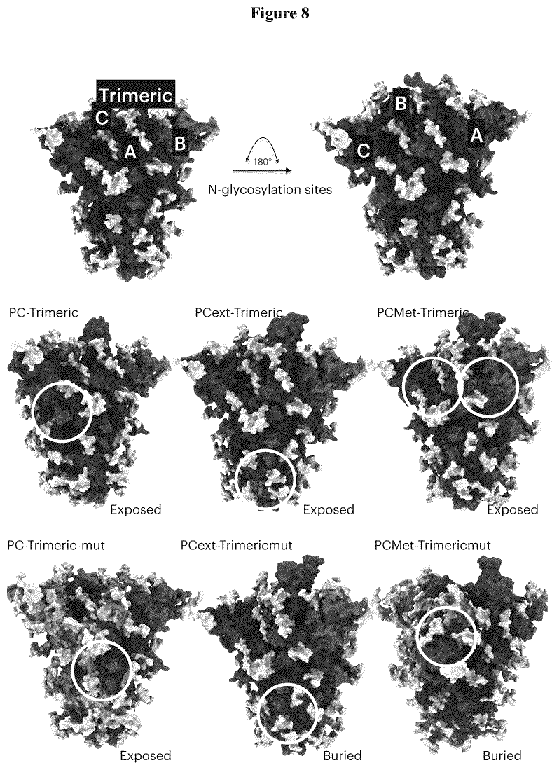

FIG. 8: Surface state analysis of the PC and associated molecules i.e., exposed and buried from sugar moieties (white circles) in trimeric wildtype and mutant forms. The circle represents the high affinity interaction sites.

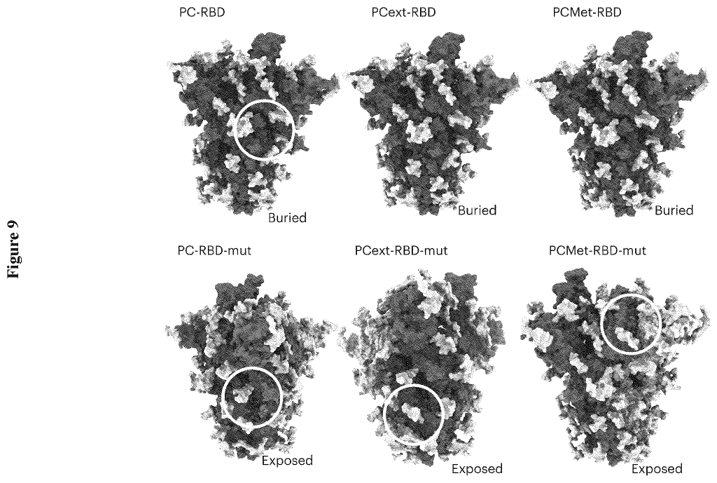

FIG. 9: Surface state analysis of the PC and associated molecules i.e., exposed and buried from sugar moieties (white circles) in RBD wildtype and mutant forms. The circle represents the high affinity interaction sites.

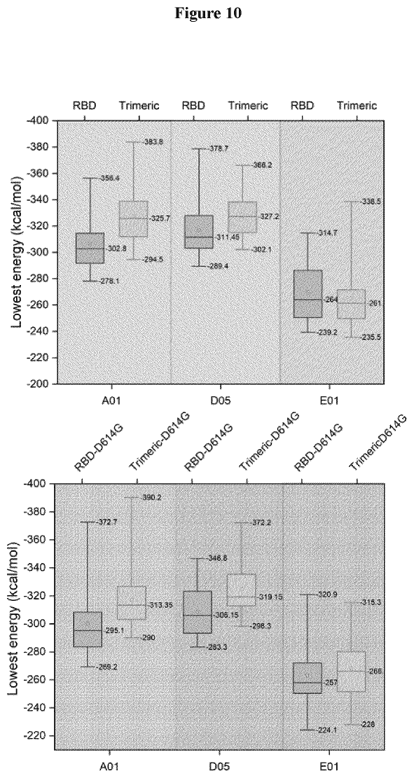

FIG. 10: Antibody-antigen interaction analysis: Box plots representing the lowest energy configurations of anti-PC clones A01, E01 and D05 with trimeric, RBD and their mutant forms.

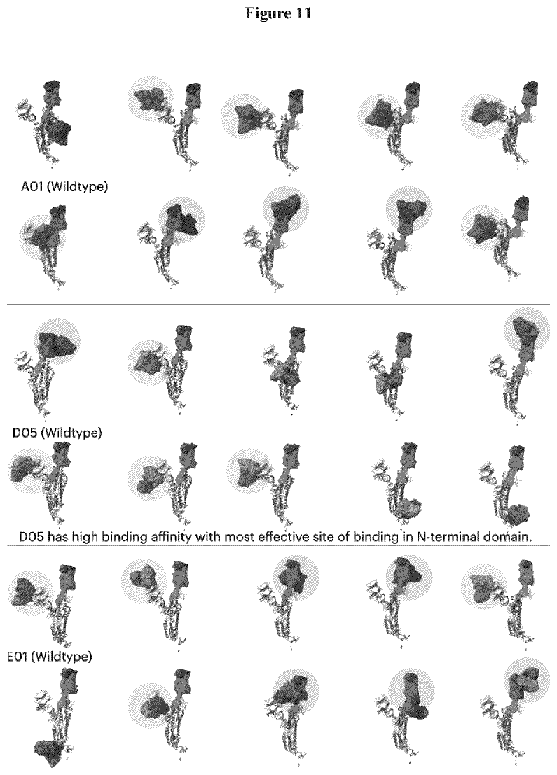

FIG. 11: Structural representation of the anti PC-RBD (wildtype). The circle represents the interaction sites.

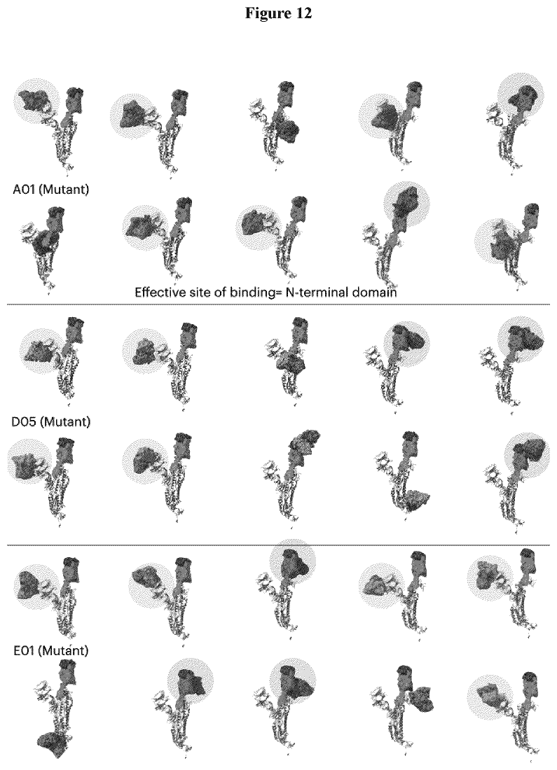

FIG. 12: Structural representation of the anti PC-RBD (mutant). The circle represents the interaction sites.

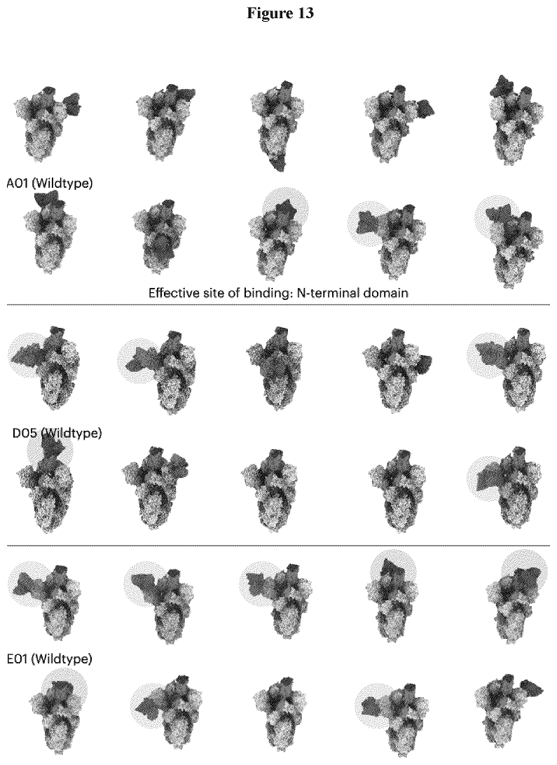

FIG. 13: Structural representation of the anti PC-trimeric (wildtype). The circle represents the interaction sites.

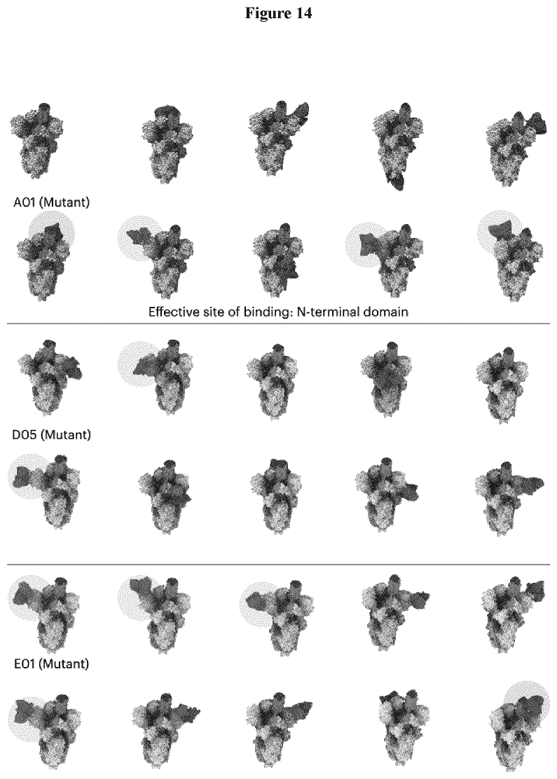

FIG. 14: Structural representation of the anti PC-trimeric (mutant). The circle represents the interaction sites.

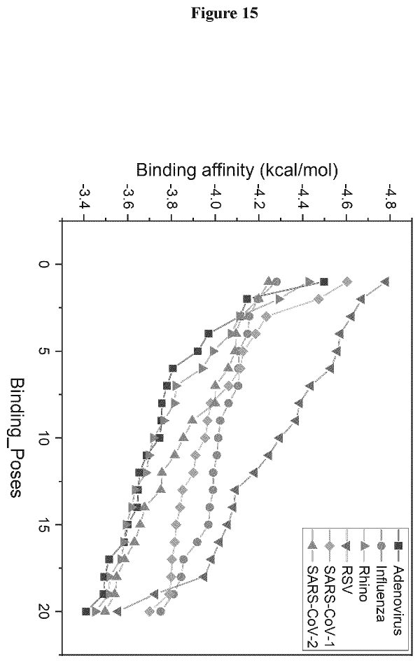

FIG. 15: Binding affinity of PC towards other virus that causes respiratory infection including SARS-CoV-2. Binding poses indicates the conformations of PC binding to these proteins.

### EXAMPLES

### Example 1: Experimental Data—Antibodies Against Phosphorylcholine are Associated with Less Severe Disease in COVID-19

### Abstract

IgM antibodies against phosphorylcholine (anti-PC) are associated with protection in atherosclerosis, cardiovascular disease (CVD), and also inflammatory and rheumatic conditions like SLE, RA and others. Underlying potential mechanisms include anti-inflammatory (protection against inflammatory and oxidised phospholipids) and promotion of T regulatory cells; inhibition of cell death, increased clearance of dead cells.

### Methods

13 individuals with diagnosed COVID-19 were included in the study. Patients were divided into less and more severe symptoms. Survival after 28 days was used as outcome. IgM anti-PC was determined by ELISA. We used a molecular docking approach, to retrieve information regarding binding properties to PC in relation to SARS-CoV-2.

### Results

IgM anti-PC was significantly lower among COVID-19 patients as compared to healthy controls, and also among those with severe disease as compared to those with less severe disease. Mean levels of IgM anti-PC were non-significantly higher among survivors as compared to those who died from the disease. By use of in silico methods, we determined that PC binds the Spike protein in SARS-CoV-2.

### Conclusions

IgM anti-PC is lower in severe COVID-19 as compared to less severe disease and also lower among COVID-19 patients than controls. Through in silico methods we determined that PC can bind to the Spike protein in SARS-CoV-2. Anti-PC therefore appears to have protective properties in COVID-19.

### Background

The virus causing COVID-19, SARS-CoV-2 has several effects on, heart, vessels, lungs and immune system causing severe cardiopulmonary disease (CPD). Firstly, a common complication in COVID-19 is damage to myocardium, with troponin release. Secondly, CPD and high age is associated with worse outcome. Thirdly, SARS-CoV-2 through the Spike protein, binds and enters cells through ACE-2, a protein with important properties in relation to both regulation of blood pressure and homeostasis, but also with anti-inflammatory properties. Fourthly, SARS-CoV-2 causes a cytokine storm in some patients, an important cause of disease severity and death. Here proinflammatory cytokines and dysregulation of these by the virus and its immune effects are underlying causes.¹

Phosphorylcholine (PC) is an important danger-associated molecular pattern (DAMP), and an antigen in oxidised low-density lipoprotein (OxLDL) where it becomes exposed on oxidised phospholipids during LDL-oxidation. OxLDL is abundant in atherosclerotic plaques together with dead cells, and OxLDL may be a cause of the inflammation typical of these lesions, activating immune competent cells including monocytes, dendritic cells (DC) and T cells. PC is also exposed on dead cells.² PC is also a pathogen-associated molecular pattern (PAMP) and an antigen on bacteria, parasites and nematodes.³ Antibodies against PC (anti-PC) are present in healthy adults, and 5-10% of circulating IgM consists of IgM anti-PC.^{2, 4, 5} IgM anti-PC is associated with protection in several chronic inflammatory disease conditions, including atherosclerosis and cardiovascular disease (CVD), rheumatic diseases and chronic kidney disease (CKD) and several potential mechanisms have been described, in this context the anti-inflammatory effects are especially interesting².

To elucidate if PC may relate to SARS-CoV-2 Spike protein, we used molecular modelling approaches including molecular docking studies to depict the binding affinity and intrinsic atomistic interactions of PC. By use of these in silico methods, links between different compounds as Spike protein and PC can be determined. We hereby report that IgM anti-PC is low in severe COVID-19 and that PC binds Spike protein in simulation models. The implications are discussed.

### Antibody Determination

IgM anti-PC were determined by ELISA. Briefly, the concentration of the antigen used in each well was 10 μg/ml. Nunc Immuno microwell plates (Thermo Labsystems, Franklin Lakes, MA, USA) were coated with PC-bovine serum albumin (BSA). Coated plates were incubated overnight at 4 C. After four washings with wash buffer (1×PBST), the plates were blocked with 2% BSA-phosphate-buffered saline (PBS) for 1 h at room temperature. Plates were again washed then the samples were diluted 1:200 times for all the antibodies in 0.2% BSA-PBS and added at 100 μl/well. Plates were incubated at room temperature for 2 h and washed as described above. Biotin-conjugated goat anti-Human IgM, (diluted 1:15 000, 1% BSA-PBS) was added at 100 μl/well and incubated at room temperature for 2 h. After four washings, the plate was incubated with horseradish peroxidase conjugated streptavidin, 1:3000 in 0.2% BSA-PBS) (Thermo Scientific, Roskilde, Denmark) at 100 μl/well for 20 mins. The color was developed by adding the horseradish peroxidase substrate, 3,3′,5,5′-tetramethylbenzidine (TMB) (3.30, 5.50; Sigma Aldrich) at 100 μl/well and incubating the plates for 5 mins, 5 mins, 15 mins, 20 mins, 5 mins respectively, at room temperature in the dark. Further reaction was stopped with stop solution 1 N H2SO4 at 50 μl/well. Finally, plates were read with the Biotek 800 TS absorbance reader at 450 and 630 nm. Delta value was determined via the subtraction of the blanked OD at 630 nm from the blanked OD at 450 nm. The Delta value of the respective sample was then divided by the Delta value of the standard to reach a relative Unit value of the abundance of antibodies in this sample. All samples were measured in duplicate within a single assay and the CV between the duplicates was below 15% for all the antibodies. Pooled serum from Sigma Aldrich (St Louis, MO, USA) was used as a standard control for each plate. A sample from a control group was used as an internal control for each plate, the ratio of Internal control and standard control was used to determine the CV between the plates. The CV between the plates was kept below 10%.

in-Silico Methods

To elucidate the clinical validations, molecular-docking approach has been taken into consideration to know the exact mechanism of PC binding to Spike protein of SARS-CoV-2. SARS-CoV-2 trimeric spike protein (PDB ID: 6VSB) was taken into account for molecular docking approach. Further, the trimeric spike protein was subjected for molecular docking using Autodock Vina (O. Trott, A. J. Olson, AutoDock Vina: improving the speed and accuracy of docking with a new scoring function, efficient optimization and multithreading, Journal of Computational Chemistry 31 (2010) 455-461) as receptor and PC parameters were set as ligand respectively. Grid dimensions was set to 126×126×126, with a spacing of 1 Å. We have used Autodock vina (v.1.2.2) algorithm to determine the binding specificity and interaction energies. The RMSD tolerance was set to 2 Å with rate of mutation 2% The post docking analysis was performed using ChimeraX (UCSF ChimeraX: Structure visualization for researchers, educators, and developers. Pettersen E F, Goddard T D, Huang C C, Meng E C, Couch G S, Croll T I, Morris J H, Ferrin T E. Protein Sci. 2021 January; 30(1):70-82) and Discovery studio Visualizer (BIOVIA, Dassault Systemes, Discovery Studio Visualizer, DS2021, San Diego: Dassault Systemes, 2021). We have not only evaluated the Phosphorylcholine specificity interactions but also for comparison Phosphatidylcholine and 2-methacryloyloxyethyl phosphorylcholine were also considered (FIG. 1). Furthermore, we have not only considered the wild type spike protein but also the RBD domain of the trimeric spike protein to elucidate the interaction mechanism. On the other hand, the mutant D614G mutation has also been considered to evaluate various conformations of the ligands binding to the trimeric and RBD domains. Moreover, we have also predicted the antibody-antigen interactions using Cluspro 2.0 (Desta I T, Porter K A, Xia B, Kozakov D, Vajda S. Performance and Its Limits in Rigid Body Protein-Protein Docking. Structure. 2020 September; 28 (9):1071-1081) to elucidate the specificity of anti-PC clones i.e., E01, A01 and D05 Fiskesund et al., 2014) binding to trimeric, RBD and their mutant forms.

### Results

We divided the COVID 19 patients in 2 groups, firstly mild (severity 1), moderate (severity 2) and secondly severe (severity 3) cases. There was a total of 134 patients measured for IgM anti-PC at different time intervals as indicated. Also, healthy controls were included.

At baseline, the differences between the groups are demonstrated (FIG. 2A). Significant differences between controls and cases were noted. When COVID-19 cases were divided between severe (severity 1+2 vs 3), IgM anti-PC antibodies were found to be significantly lower in severity 3 group compared to severity group 1+2 (p<0.02). IgM anti-PC antibodies were also found to be significantly lower in the severity 3 group compared to healthy controls (p<0.05).

We compared the difference between anti-PC antibody profile of COVID-19 patients who died within 28 days after being included in the study. Even though the median level of IgM anti-PC was lower among non-survivors, this difference did not reach statistical significance (FIG. 2B).

Binding Mechanism of PC, Phosphatidylcholine and 2-Methacryloyloxyethyl Phosphorylcholine with Trimeric SARS-CoV-2 and RBD Along with the Mutant D614G

The probable mechanism behind the hijacking of interaction between ACE2 and trimeric Spike glycoprotein has been depicted in FIG. 1C. The mechanism depicts that when PC binds to specific binding sites of trimeric S-protein, it triggers the anti-PC molecules to binds to the ACE2 interaction specific binding site (RBD domain of S-protein) and hijack the entry to the host cells. Site-specific binding may also play a pivotal role in deciphering the action triggered by PC molecules. Therefore, we have focused our analysis towards In-silico methods (molecular modelling and docking approach) to evaluate the intrinsic atomistic interaction of PC and associated molecules with trimeric S protein, RBD, mutant D614G and further we have compared with site specific glycosylated S-protein (PDB ID: 6VSB). FIG. 3 illustrates the binding affinities of PC and associated molecules with RBD, trimeric spike and their mutant forms.

In order to evaluate the PC and the associated molecules binding sites with the glycosylated S protein, we superimpose the glycosylated molecules to the native conformation of the S-protein and showed that the binding orientations. Generally, these glycan sites clusters to shield their active and interaction sites from drugs, vaccines and antibodies. As shown in FIG. 3, in both wild type and mutant forms of RBD and trimeric spike, Phosphatidylcholine has more binding affinity in comparison to PC and methylated PC. This was again confirmed with predictive values obtained from inhibition constant. The binding affinity of Phosphatidylcholine (PC-extd) was in the range of −4.5 to −5.5 kcal/mol. Although there was not much difference in binding affinities among the molecules but was drastically impacted with the mutant forms. D614G mutation does have significant effect on binding energies, and it can be predicted that the mutant forms enable all the PC associated molecules to bind with higher affinity.

The binding conformations were again visually inspected to identify the sites where these molecules bind to RBD, Trimeric and their mutant forms. In case of RBD, PC binds to the RBD domain, PC (extd) binds at the end points of RBD and the methylated PC binds to the N-terminal domain (also observed in case of PC as well). Whereas in the mutant form, PC and all the other molecules binds mostly to N-terminal domain. This means that the N-terminal domain have site specific interaction and affinities toward molecules in mutant form (FIG. 4).

In case of trimeric wildtype and mutant, only PC-extd and methylated PC binds to RBD domains but PC was mostly distributed along the surface of trimeric wildtype. In case of mutant trimeric, again N-terminal domain has an effect of interaction sites (FIG. 5).

To understand the overall picture, we have also represented here interaction sites of all the molecules in RBD, trimeric and mutant forms. In RBD, the molecules bind to all the sites and spread across all the sites whereas in mutant form, most of the molecules bind to N-terminal region (FIG. 6).

In trimeric form, the molecules interactions are mostly at the RBD and S1/S2 cleavage sites whereas in mutant form, most of the molecules bind to N-terminal region. (FIG. 7).

The Spike protein of the novel coronavirus SARS-CoV2 contains an insertion at the boundary of S1/S2 subunits forming a cleavage motif RxxR for furin-like enzymes. Cleavage at S1/S2 is important for efficient viral entry into target cells. Furthermore, S2 subunit plays a key role in mediating virus fusion with and entry into the host cell, in which the heptad repeats 1 (HR1) and heptad repeat 2 (HR2) can interact to form six-helical bundle (6-HB), thereby bringing viral and cellular membranes in close proximity for fusion.

Furthermore, we have done analysis on buried and exposed sites of the molecules binding to the trimeric and RBD along with their mutant forms. As reported earlier, N-linked glycosylated sites of trimeric S-protein in both up and down conformations hinder many drugs and antibodies to bind to the receptor binding sites (enables entry to the host cells) which is a typical shielding mechanism of all viral proteins (Watanabe et al. (2020), Science, 369: 6501; DOI 10.1136/science.abb9983; https://science.sciencemag.org/content/369/6501/330). In case of wildtype, all the interaction sites were exposed and not shielded by the sugar moieties. In mutant trimeric, only PC is exposed on the surface, but all the other molecules were buried and shielded by sugar moieties (FIG. 8). In case of RBD wildtype, all the molecules were buried but in mutant form, all the molecules were not shielded by sugar moieties (FIG. 9).

Anti-PC Clones' Effect on Trimeric Spike, RBD and their Mutant Form D614G

We have evaluated the antibody-antigen interaction to assess the specificity of anti-PC clones that were generated inhouse i.e. A01, E01 and D05 clones (FIG. 10).

From the interaction analysis, it was depicted that in wildtype form of RBD, D05 tends to have high affinity whereas in trimeric, A01 has high affinity based on the lowest energy configurations. In case of mutant form, A01 outperforms all the other clones in terms of affinity. The specificity of the three clones with variation in binding affinity tends to show that the three clones have selectivity criteria based on the ratio of the affinity of the compound towards the off-target protein relative to the target protein. The larger the Kd ratio, the better the selectivity. (Kd ratio=Kd,off target/Kd,target). Also, it depends on the non-polar group present on the target. The energy thus obtained from the analysis have been evaluated based on the following criteria:

$$E = 0.50 {E}_{rep} + - 0.2 {E}_{a t t} + 600 {E}_{e l e c} + 0.25 {E}_{DARS}$$

Eᵣₑₚ and Eₐₜₜᵣ denote the repulsive and attractive contributions to the van der Waals interaction energy, and E_{elec} is an electrostatic energy term. EDARS is a pairwise structure-based potential constructed by the Decoys as the Reference State (DARS) approach, and it primarily represents desolvation contributions, i.e., the free energy changes due to the removal of the water molecules from the interface. Graphical representation of the anti-PC binding to the wildtype RBD, trimeric and their mutant forms have been depicted in FIG. 11-14).

Binding Affinity of PC Towards Other Virus that Causes Respiratory Infection Including SARS-CoV-2

We have considered 5 other virus surface proteins including SARS-CoV-2 spike protein to predict the binding affinity of PC.

### 1. Adenovirus: Uniprot ID: P03252

Cleaves viral precursor proteins (pTP, pIIIa, pVI, pVII, pVIII, and pX) inside newly assembled particles giving rise to mature virions. Protease complexed to its cofactor slides along the viral DNA to specifically locate and cleave the viral precursors. Mature virions have a weakened organisation compared to the unmature virions, thereby facilitating subsequent uncoating. Without maturation, the particle lacks infectivity and is unable to uncoat. Late in adenovirus infection, in the cytoplasm, may participate in the cytoskeleton destruction.

### 2. Influenza: Uniprot ID: C3W5S1

Binds to sialic acid-containing receptors on the cell surface, bringing about the attachment of the virus particle to the cell. This attachment induces virion internalisation either through clathrin-dependent endocytosis or through clathrin- and caveolin-independent pathway. Plays a major role in the determination of host range restriction and virulence. Responsible for penetration of the virus into the cell cytoplasm by mediating the fusion of the membrane of the endocytosed virus particle with the endosomal membrane. Low pH in endosomes induces an irreversible conformational change in HA2, releasing the fusion hydrophobic peptide.

### 3. Rhinovirus Uniprot ID: Q82122

Rhinoviruses belong to the picornavirus family and are small, icosahedral, non-enveloped viruses containing one positive RNA strand. Human rhinovirus 16 (HRV16) belongs to the major receptor group of rhinoviruses, for which the cellular receptor is intercellular adhesion molecule-1 (ICAM-1). In many rhinoviruses, one of the viral coat proteins (VP1) contains a hydrophobic pocket which is occupied by a fatty acid-like molecule, or so-called ‘pocket factor’. Antiviral agents have been shown to bind to the hydrophobic pocket in VP1, replacing the pocket factor. The presence of the antiviral compound blocks uncoating of the virus and in some cases inhibits receptor attachment.

### 4. RSV Uniprot ID: O36634

A precursor that is cleaved at two sites by a furin-like protease to give rise to the mature F1 and F2 fusion glycoproteins. The F glycoprotein is synthesised as a F0 inactive precursor that is heavily N-glycosylated and processed at two sites by a host furin-like protease probably in the Golgi. The cleavage site between p27 and F1 may occur after endocytosis to yield the mature F1 and F2 proteins. Both cleavages are required for membrane fusion and p27 is released from the processed protein.

### 5. SARS-CoV-1 Uniprot ID: P59594

Viral protein involved in the merging of the virus envelope with host endosomal membrane during viral penetration into host cell. Viral fusion proteins drive this fusion reaction by undergoing a major conformational change that is triggered by interactions with the target cell. The specific trigger is mainly endosome acidification which induce activation of the fusion protein by conformational change.

### 6. SARS-CoV-2 Uniprot ID: P0DTC2

Attaches the virion to the cell membrane by interacting with host receptor, initiating the infection. Binding to human ACE2 receptor and internalisation of the virus into the endosomes of the host cell induces conformational changes in the Spike glycoprotein

Using molecular docking approach, we have predicted the binding affinity of PC with the viruses listed above. We can decipher from the analysis that all the respiratory viral surface proteins bind to PC with greater affinity which may elicit the immune response. The binding affinity is as follows RSV>SARS-CoV-1>Adenovirus>Rhino>Influenza>SARS-CoV-2. As you can see from the analysis that Influenza virus and SARS-CoV-2 has similar binding affinity in comparison to other viruses. A clinical study has been proposed here: Seets et al. (2022), Lancet, 399:10334, pages 1463-1464 https://www.thelancet.com/journals/lancet/article/PIIS0140-6736(22)00383-X/fulltext).

### Discussion

We hereby report that IgM anti-PC is lower among COVID-19 patients with severe disease, than among those with less severe disease. Further, low levels of IgM anti-PC (being in the lowest tertile of anti-PC levels) was more common among those with more severe disease.

The abovementioned results indicate that low levels of anti-PC antibodies could be considered a contributing cause of complications including inflammation in viral diseases. This is particularly relevant to Covid-19, where hyper-inflammatory responses cause a cytokine storm in some patients, which is an important contributor to disease severity and death. The inventors have made a crucial contribution by identifying the therapeutic potential anti-PC antibodies to prevent and/or treat severe viral disease, and by providing approaches for identifying individuals at risk of severe viral disease and hospitalisation.

We have previously demonstrated that IgM anti-PC is associated with protection in atherosclerosis progress, CVD, chronic kidney disease (CKD).^{2, 8-12} In the context of COVID-19 and inflammatory complications, it is interesting to note that also in autoimmune diseases, IgM anti-PC is a protection marker, especially in SLE^{10, 13} and other systemic autoimmune conditions¹⁴, but also in RA, where low levels of these antibodies predict being a non-responder to biologics.¹⁵ These findings have been largely confirmed by other groups¹⁶⁻²², and extended to other chronic inflammatory diseases, including vasculitis¹⁹ and osteoarthritis²¹.

In line with this are animal experiments, which support a protective role of anti-PC against atherosclerosis²³, SLE²⁴, and RA²⁵. In a recent study, we studied that brown bears (Ursus arctos) which do not develop signs of atherosclerosis in spite of having high blood lipids during hibernation when they also are immobilised and uremic. We suggested that the high anti-PC levels they have during hibernation could contribute to protection and may be related to exposure to different microorganisms during fall when they consume huge amounts of different types of nutrients.²⁶

Of note, even though we cannot exclude consumption of anti-PC low levels of anti-PC irrespective of cause could still have implications for disease severity. Several potential mechanisms have been described. This most important in the context of COVID-19 is likely to be an anti-inflammatory. Anti-PC inhibits the effects of oxidised and proinflammatory phospholipids, related to oxLDL and/or platelet activating factor (both of which have PC as a major antigen in this context.¹¹ this effect could be highly relevant in a situation with oxidative stress and increased lipid peroxidation, which is known to be implicated in different viral diseases, including influenza, and also COVID-19 and may be a contributing cause of pathogenesis, immune dysfunction, apoptosis and inflammation. In general, oxidative stress is caused by an imbalance between pro- and antioxidant mechanisms, which promotes lipid- and DNA-oxidation, damage and interestingly, viral infections are associated with decreased antioxidant defences.^{27, 28} Low levels of anti-PC could thus promote a proinflammatory state. Another anti-inflammatory mechanism is promotion of polarisation of T regulatory cells. IgM anti-PC increased significantly the proportion of Tregs from healthy donors, SLE patients and atherosclerotic plaque T cells.²⁹ In a recent systematic search 18 studies were identified where Tregs were studied, and a trend toward decreasing Tregs was reported in COVID-19, which suggests that Tregs may be of major importance in COVID-19 to prevent severe disease development.³⁰

Another mechanism we reported is inhibition of cell death caused by an important product of lipid oxidation, lysophosphatidylcholine (LysoPC)³¹ and since it is possible that tissue damage due to oxidative stress could play a role in COVID-19, also this mechanism may have some protective role in COVID-19. Inhibition by IgM anti-PC of uptake of oxLDL in macrophages³², turning them into inert foam cells which stay in plaques and constitute much of the central core of death cells, may not be so important in COVID-19 though. But clearance of dead cells, which is a feature of IgM anti-PC⁴, could play a role, since cell death is a major feature of T cell responses (which attack infected cells) and clearance of dead cells is important to diminish the proinflammatory load. Anti-PC, especially IgM, IgG1 and IgA anti-PC, could thus be protective in COVID-19 through different non-mutually exclusive properties.

We believe that, in a development of the Hygiene/Old Friends hypothesis that lack of exposure microorganisms including nematodes, parasites, and also bacteria (including Treponema) which have PC as an antigen, could cause low levels of anti-PC and thus increased risk of inflammatory conditions.^{2, 33-35} Low levels of anti-PC could be seen as an immune-deficient state, predisposing also to more severe COVID-19.

Anti-PC levels are higher in women than men in all studies we are aware of. This was also the case individuals leading a life as hunters, gatherers and horticulturalists, from Kitava, New Guinea in the 90s.^{2, 33-35} A major feature of COVID-19 is that it affects men more severely than women. The higher levels of anti-PC in women may explain the fact that women are less severely affected by COVID-19.

There were fewer women than men in this study, and individuals with severe COVID-19 were older than those with less severe disease. This is a well-known feature of COVID-19, though the causes are not clear, and several non-mutually exclusive possibilities have been proposed. We then asked whether PC could interact with and/or bind to SARS-CoV-2, by simulation of biological pathways in relation to the Spike-protein with focus also on PC. Such approach can be used both for explorative studies and drug development, where an important consideration is the complexity of interaction networks, which also include the target protein neighborhood and the disease can be an outcome of multiple layers of interaction. A number of databases where proteome broad protein-chemical interactions can be used, and many protein-chemical interactions are essential to discern protein-chemical interaction networks.^{36, 37}. By use of such bioinformatics-related methods, we hereby report for the first time that PC interacts strongly with the Spike protein in SARS-CoV-2-virus. This provides a mechanistic explanation to the other findings herein. This structural finding coupled with the clinical observations that IgM anti-PC antibodies are lower in mild and moderate and severe COVID-19 patient shows that anti-PC antibodies are acting against virus infections, by binding and neutralising and/or inhibiting the effects of SARS-CoV-2 and other viruses binding to their targets.

Taken together, we report that IgM anti-PC is lower in severe COVID-19. By bioinformatics-related methods we demonstrate that PC binds to Spike protein of SARS-CoV-2 which indicates that anti-PC may act against the virus.

### REFERENCES

1. Tang L, Yin Z, Hu Y and Mei H. Controlling Cytokine Storm Is Vital in COVID-19. Front Immunol. 2020; 11:570993.
2. Frostegard J. Immunity, atherosclerosis and cardiovascular disease. BMC Med. 2013; 11:117.
3. Harnett W and Harnett M M. Phosphorylcholine: friend or foe of the immune system? Immunol Today. 1999; 20:125-9.
4. Rahman M, Sing S, Golabkesh Z, Fiskesund R, Gustafsson T, Jogestrand T, Frostegård AG, Hafström I, Liu A and Frostegård J. IgM antibodies against malondialdehyde and phosphorylcholine are together strong protection markers for atherosclerosis in systemic lupus erythematosus: Regulation and underlying mechanisms. Clin Immunol. 2016; 166-167:27-37.
5. Thiagarajan D, Frostegard A G, Singh S, Rahman M, Liu A, Vikstrom M, Leander K, Gigante B, Hellenius M L, Zhang B, Zubarev R A, de Faire U, Lundstrom S L and Frostegard J. Human IgM Antibodies to Malondialdehyde Conjugated With Albumin Are Negatively Associated With Cardiovascular Disease Among 60-Year-Olds. J Am Heart Assoc. 2016; 5.
6. Szklarczyk D, Santos A, von Mering C, Jensen U, Bork P and Kuhn M. STITCH 5: augmenting protein-chemical interaction networks with tissue and affinity data. Nucleic Acids Res. 2016; 44:D380-4.
7. Kuhn M, von Mering C, Campillos M, Jensen L J and Bork P. STITCH: interaction networks of chemicals and proteins. Nucleic Acids Res. 2008; 36:D684-8.
8. Caidahl K, Hartford M, Karlsson T, Herlitz J, Pettersson K, de Faire U and Frostegard J. IgM-phosphorylcholine autoantibodies and outcome in acute coronary syndromes. Int J Cardiol. 2013; 167:464-9.
9. Su J, Georgiades A, Wu R, Thulin T, de Faire U and Frostegard J. Antibodies of IgM subclass to phosphorylcholine and oxidized LDL are protective factors for atherosclerosis in patients with hypertension. Atherosclerosis. 2006; 188:160-6.
10. Anania C, Gustafsson T, Hua X, Su J, Vikstrom M, de Faire U, Heimburger M, Jogestrand T and Frostegård J. Increased prevalence of vulnerable atherosclerotic plaques and low levels of natural IgM antibodies against phosphorylcholine in patients with systemic lupus erythematosus. Arthritis Res Ther. 2010; 12:R214.
11. Su J, Hua X, Concha H, Svenungsson E, Cederholm A and Frostegård J. Natural antibodies against phosphorylcholine as potential protective factors in SLE. Rheumatology (Oxford). 2008; 47:1144-50.
12. Carrero J J, Hua X, Stenvinkel P, Qureshi A R, Heimburger O, Barany P, Lindholm B and Frostegard J. Low levels of IgM antibodies against phosphorylcholine-A increase mortality risk in patients undergoing haemodialysis. Nephrol Dial Transplant. 2009; 24:3454-60.
13. Su J, Hua X, Concha H, Svenungsson E, Cederholm A and Frostegard J. Natural antibodies against phosphorylcholine as potential protective factors in SLE. Rheumatology (Oxford). 2008; 47:1144-50.
14. Thiagarajan D, Oparina N, Lundstrom S, Zubarev R, Sun J, Consortium P C, Alarcon-Riquelme M and Frostegard J. IgM antibodies against malondialdehyde and phosphorylcholine in different systemic rheumatic diseases. Sci Rep. 2020; 10:11010.
15. Ajeganova S, Fiskesund R, de Faire U, Hafstrom I and Frostegard J. Effect of biological therapy on levels of atheroprotective antibodies against phosphorylcholine and apolipoproteins in rheumatoid arthritis—a one year study. Clin Exp Rheumatol. 2011; 29:942-50.
16. Vas J, Gronwall C, Marshak-Rothstein A and Silverman G J. Natural antibody to apoptotic cell membranes inhibits the proinflammatory properties of lupus autoantibody immune complexes. Arthritis Rheum. 2012; 64:3388-98.
17. Sobel M, Moreno K I, Yagi M, Kohler T R, Tang G L, Clowes A W, Zhou X H and Eugenio E. Low levels of a natural IgM antibody are associated with vein graft stenosis and failure. J Vasc Surg. 2013; 58:997-1005 e1-2.
18. Gleissner C A, Erbel C, Haeussler J, Akhavanpoor M, Domschke G, Linden F, Doesch A O, Conradson G, Buss S J, Hofmann N P, Gitsioudis G, Katus H A and Korosoglou G. Low levels of natural IgM antibodies against phosphorylcholine are independently associated with vascular remodeling in patients with coronary artery disease. Clinical research in cardiology: official journal of the German Cardiac Society. 2015; 104:13-22.
19. Wilde B, Slot M, van Paassen P, Theunissen R, Kemna M, Witzke O and Cohen Tervaert J W. Phosphorylcholine antibodies are diminished in ANCA-associated vasculitis. Eur J Clin Invest. 2015; 45:686-91.
20. Imhof A, Koenig W, Jaensch A, Mons U, Brenner H and Rothenbacher D. Long-term prognostic value of IgM antibodies against phosphorylcholine for adverse cardiovascular events in patients with stable coronary heart disease. Atherosclerosis. 2015; 243:414-20.
21. Nguyen T G, McKelvey K J, March L M, Hunter D J, Xue M, Jackson C J and Morris J M. Aberrant levels of natural IgM antibodies in osteoarthritis and rheumatoid arthritis patients in comparison to healthy controls. Immunol Lett. 2016; 170:27-36.
22. Lopez P, Rodriguez-Carrio J, Martinez-Zapico A, Perez-Alvarez A I, Benavente L, Caminal-Montero L and Suarez A. IgM anti-phosphorylcholine antibodies associate with senescent and IL-17+ T cells in SLE patients with a pro-inflammatory lipid profile. Rheumatology (Oxford). 2019.
23. Caligiuri G, Khallou-Laschet J, Vandaele M, Gaston A T, Delignat S, Mandet C, Kohler H V, Kaveri S V and Nicoletti A. Phosphorylcholine-targeting immunization reduces atherosclerosis. J Am Coll Cardiol. 2007; 50:540-6.
24. Aprahamian T R, Zhong X, Amir S, Binder C J, Chiang L K, Al-Riyami L, Gharakhanian R, Harnett M M, Harnett W and Rifkin I R. The immunomodulatory parasitic worm product ES-62 reduces lupus-associated accelerated atherosclerosis in a mouse model. International journal for parasitology. 2015; 45:203-7.
25. Chen Y, Khanna S, Goodyear C S, Park Y B, Raz E, Thiel S, Gronwall C, Vas J, Boyle D L, Corr M, Kono D H and Silverman G J. Regulation of dendritic cells and macrophages by an anti-apoptotic cell natural antibody that suppresses TLR responses and inhibits inflammatory arthritis. J Immunol. 2009; 183:1346-59.
26. Samal S K, Frobert O, Kindberg J, Stenvinkel P and Frostegard J. Potential natural immunization against atherosclerosis in hibernating bears. Sci Rep. 2021; 11:12120.
27. Chernyak B V, Popova E N, Prikhodko A S, Grebenchikov O A, Zinovkina L A and Zinovkin R A. COVID-19 and Oxidative Stress. Biochemistry (Mosc). 2020; 85:1543-1553.
28. Laforge M, Elbim C, Frere C, Hemadi M, Massaad C, Nuss P, Benoliel J J and Becker C. Tissue damage from neutrophil-induced oxidative stress in COVID-19. Nat Rev Immunol. 2020; 20:515-516.
29. Sun J, Lundstrom S L, Zhang B, Zubarev R A, Steuer J, Gillgren P, Rahman M, Ajeganova S, Liu A and Frostegard J. IgM antibodies against phosphorylcholine promote polarization of T regulatory cells from patients with atherosclerotic plaques, systemic lupus erythematosus and healthy donors. Atherosclerosis. 2018; 268:36-48.
30. Rahimzadeh M and Naderi N. Toward an understanding of regulatory T cells in COVID-19: A systematic review. J Med Virol. 2021.
31. Fiskesund R, Su J, Bulatovic I, Vikstrom M, de Faire U and Frostegard J. IgM phosphorylcholine antibodies inhibit cell death and constitute a strong protection marker for atherosclerosis development, particularly in combination with other auto-antibodies against modified LDL. Results Immunol. 2012; 2:13-8.
32. de Faire U, Su J, Hua X, Frostegard A, Halldin M, Hellenius M L, Wikstrom M, Dahlbom I, Gronlund H and Frostegard J. Low levels of IgM antibodies to phosphorylcholine predict cardiovascular disease in 60-year old men: effects on uptake of oxidized LDL in macrophages as a potential mechanism. J Autoimmun. 2010; 34:73-9.
33. Frostegård J, Tao W, Rastam L, Lindblad U and Lindeberg S. Antibodies against Phosphorylcholine among New Guineans Compared to Swedes: An Aspect of the Hygiene/Missing Old Friends Hypothesis. Immunol Invest. 2016:1-11.
34. Agmon-Levin N, Bat-sheva P K, Barzilai O, Ram M, Lindeberg S, Frostegard J and Shoenfeld Y. Antitreponemal antibodies leading to autoantibody production and protection from atherosclerosis in Kitavans from Papua New Guinea. Ann N Y Acad Sci. 2009; 1173:675-82.
35. Frostegard J, Tao W, Georgiades A, Rastam L, Lindblad U and Lindeberg S. Atheroprotective natural anti-phosphorylcholine antibodies of IgM subclass are decreased in Swedish controls as compared to non-westernized individuals from New Guinea. Nutr Metab (Lond). 2007; 4:7.
36. Sharan R, Ulitsky I and Shamir R. Network-based prediction of protein function. Mol Syst Biol. 2007; 3:88.
37. Hopkins A L. Network pharmacology: the next paradigm in drug discovery. Nat Chem Biol. 2008; 4:682-90.
38. Vaughan, T., Osbourn, J. & Tempest, P. Human antibodies by design. Nat Biotechnol. 1998; 16:535-539.
39. Binder, C. J., Shaw P. X., Chang M. K., Boullier A., Hartvigsen K., Horkko S., Miller Y. I., Woelkers D. A., Corr M., Witztum J. L. The role of natural antibodies in atherogenesis. J Lipid Res. 2005; 46(7):1353-1363.
40. Chang, M. K., Binder C. J., Miller Y. I., Subbanagounder G., Silverman G. J., Berliner J. A., Witztum J. L. Apoptotic cells with oxidation-specific epitopes are immunogenic and proinflammatory. J Exp Med. 2004; 200(11): 1359-1370.
41. Boullier, A., Gillette K. L., Horkko S., Green S. R., Friedman P., Dennis E. A., Witztum J. L., Steinberg D., Quehenberger O. The binding of oxidized low density lipoprotein to mouse CD36 is mediated in part by oxidized phospholipids that are associated with both the lipid and protein moieties of the lipoprotein. J Biol. Chem. 2000; 275(13): 9163-9169.
42. Briles D. E, Forman C., Hudak S., Claflin J. L. Anti-phosphorylcholine antibodies of the T15 idiotype are optimally protective against Streptococcus pneumoniae. J Exp Med. 1982; 156(4):1177-1185.
43. Spira G., Aguila H. L., Scharff M. D. J. Immunology. 1988; 140 (8):2675-2680.
44. Fiskesund R., Steen J., Amara K., Murray F., Szwajda A., Liu A., Douagi I., Malmström V., Frostegård J. Naturally Occurring Human Phosphorylcholine Antibodies Are Predominantly Products of Affinity-Matured B Cells in the Adult. J Immunol. 2014; 192(10):4551-4559.
45. Gora S., Maouche S., Atout R., Wanherdrick K., Lambeau G., Cambien F., Ninio E., Karabina S. A. Phospholipolyzed LDL induces an inflammatory response in endothelial cells through endoplasmic reticulum stress signaling. FASEB Journal. 2010; 24(9):3284-3297.
46. Langer R. New methods of drug delivery. Science. 1990; 249(4976):1527-33.
47. Wrapp D., Wang N., Corbett K. S., Goldsmith J. A., Hsieh C. L., Abiona O., Graham B. S., McLellan J. S. Cryo-E M structure of the 2019-nCoV spike in the prefusion conformation. Science. 2020; 367(6483):1260-1263.
48. Walls A. C., Park Y. J., Tortorici M. A., Wall A., McGuire A. T., Veesler D. Structure, Function, and Antigenicity of the SARS-CoV-2 Spike Glycoprotein. Cell. 2020; 181(2):281-292.
49. Akpinar S, Oran M, Doğan M, Çelikkol A, Erdem I, Turgut B. The role of oxidized phospholipids in COVID-19-associated hypercoagulopathy. Eur Rev Med Pharmacol Sci. 2021; 25:5304-5309.
50. Trott, A. J. Olson, AutoDock Vina: improving the speed and accuracy of docking with a new scoring function, efficient optimization and multithreading, Journal of Computational Chemistry 31 (2010) 455-461.
51. Pettersen E F, Goddard T D, Huang C C, Meng E C, Couch G S, Croll T I, Morris J H, Ferrin T E. UCSF ChimeraX: Structure visualization for researchers, educators, and developers. Protein Sci. 2021 January; 30(1):70-82.
52. BIOVIA, Dassault Systemes, Discovery Studio Visualizer, DS2021, San Diego: Dassault Systèmes, 2021.
53. Desta I T, Porter K A, Xia B, Kozakov D, Vajda S. Performance and Its Limits in Rigid Body Protein-Protein Docking. Structure. 2020 September; 28 (9):1071-1081.

1. A protein complex comprising a carrier protein and phosphorylcholine, or an anti-phosphorylcholine antibody which binds specifically to a complex comprising a virus protein and phosphorylcholine, for use in treating and/or preventing a virus infection in an individual.

2. Use of a protein complex comprising a carrier protein and phosphorylcholine, or an anti-phosphorylcholine antibody which binds specifically to a complex comprising a virus protein and phosphorylcholine, in the manufacture of a medicament for use in treating and/or preventing a virus infection in an individual.

3. A method for treating and/or preventing a virus infection in an individual, the method comprising administering to the individual an effective amount of a protein complex comprising a carrier protein and phosphorylcholine, or an anti-phosphorylcholine antibody which binds specifically to a complex comprising a virus protein and phosphorylcholine.

4. The protein complex for use according to claim 1, or the use according to claim 2, or the method according to claim 3, wherein the carrier protein is selected from the group comprising: a virus protein, a bacterial protein.

5. The protein complex for use according to claim 1 or 4, or the use according to claim 2 or 4, or the method according to claim 3 or 4, wherein the protein complex induces and/or increases the presence and/or amount of anti-phosphorylcholine antibodies in the individual.

6. A method for predicting the prognosis of a virus infection in an individual, the method comprising the steps of:
(a) providing a test sample from the individual;
(b) determining the presence and/or amount of anti-phosphorylcholine antibodies in the test sample; and
(c) predicting the prognosis of the virus infection in the individual on the basis of the determination in Step (b).

7. Use of anti-phosphorylcholine antibodies for predicting the prognosis of a virus infection in an individual.

8. The protein complex for use according to claim 1 or 4-5, or the use according to claim 2 or 4-5, or the method according to claim 3-5, or the method according to claim 6, or the use according to claim 7, wherein the virus is selected from the group comprising: Orthomyxovirus, such as influenza virus, isavirus or thogotovirus; Parvovirus, such as adeno-associated virus (AAV) or a recombinant adeno-associated virus (rAAV); Adenovirus, such as adenovirus; Pneumovirus, such as Respiratory Syncytial Virus (RSV); Herpesvirus, such as Herpes Simplex Virus (HSV); Rhabdovirus, such as Vesicular Stomatitis Virus or Maraba Virus; Retrovirus, such as lentivirus or retrovirus, such as gamma retrovirus; Poxvirus, such as vaccinia virus; Paramyxovirus, such as Measles virus or Newcastle Disease virus; Reovirus, such as rotavirus or reovirus; Picornavirus, such as Rhinovirus, Type I Poliovirus, Coxsackievirus (such as Coxsackievirus A21) or Seneca Valley virus; Flavivirus, such as Dengue virus, Yellow fever virus, West Nile virus or Zika virus; Togavirus, such as Alphavirus; Coronavirus, such as SARS-CoV-1 or SARS-CoV-2.

9. The protein complex for use, or the use, or the method according to claim 8, wherein the virus is coronavirus, and wherein the coronavirus is selected from the group comprising: 229E (alpha coronavirus); NL63 (alpha coronavirus); OC43 (beta coronavirus); HKU1 (beta coronavirus); MERS-CoV (the beta coronavirus that causes Middle East Respiratory Syndrome, or MERS); Sars-CoV or Sars-CoV-1 (the beta coronavirus that causes severe acute respiratory syndrome, or Sars); Sars-CoV-2 (the novel coronavirus that causes coronavirus disease 2019, or COVID-19).

10. The protein complex for use, or the use, or the method according to claim 8, wherein the virus is influenza virus, and wherein the influenza virus is selected from the group comprising: Influenza A virus (IAV), Influenza B virus (IBV), Influenza C virus (ICV), Influenza D virus (IDV).

11. The protein complex for use, or the use, or the method according to any preceding claim, wherein the anti-phosphorylcholine antibodies bind specifically to a complex comprising a virus protein and phosphorylcholine.

12. The protein complex for use, or the use, or the method according to claim 11, wherein the anti-phosphorylcholine antibodies bind to the complex with a binding affinity from about −400 kcal/mol to about −200 kcal/mol.

13. The protein complex for use, or the use, or the method according to any preceding claim, wherein the virus protein is a spike protein of a coronavirus, or a portion or variant thereof.

14. The protein complex for use, or the use, or the method according to claim 13, wherein the portion is the receptor binding domain (RBD), or a portion or variant thereof.

15. The protein complex for use, or the use, or the method according to claim 13 or 14, wherein the variant comprises a D614G substitution.

16. The protein complex for use, or the use, or the method according to any of claims 1-12, wherein the virus protein is the hemagglutinin protein of influenza virus, or a portion or variant thereof.

17. The protein complex for use, or the use, or the method according to any of claims 1-12, wherein the virus protein is the L3 protein of adenovirus, or a portion or variant thereof.

18. The protein complex for use, or the use, or the method according to any of claims 1-12, wherein the virus protein is the genome polyprotein of rhinovirus.

19. The protein complex for use, or the use, or the method according to any of claims 1-12, wherein the virus protein is a fusion glycoprotein of respiratory syncytial virus.

20. The protein complex for use, or the use, or the method according to any preceding claim, wherein the anti-phosphorylcholine antibodies reduce and/or inhibit the binding of the virus to its target cell.

21. The protein complex for use, or the use, or the method according to any preceding claim, wherein the anti-phosphorylcholine antibodies elicit an immune response, optionally leading to virus clearance.

22. The protein complex for use, or the use, or the method according to any preceding claim, wherein the anti-phosphorylcholine antibodies inhibit the effects of oxidised and/or proinflammatory phospholipids.

23. The protein complex for use, or the use, or the method according to any preceding claim, wherein the phosphorylcholine binds to the virus protein, or a portion or variant thereof with a binding affinity from about −5.75 kcal/mol to about −2.75 kcal/mol.

24. The protein complex for use, or the use, or the method according to any preceding claim, wherein the phosphorylcholine binds to the virus protein, or a portion or variant thereof with an inhibition constant (Kᵢ) from about 0 mM to about 5 mM.

25. The protein complex for use, or the use, or the method according to any preceding claim, wherein binding of the phosphorylcholine to the virus protein permits virus entry into a target cell.

26. The protein complex for use, or the use, or the method according to any preceding claim, wherein the anti-phosphorylcholine antibodies are IgM, IgG and/or IgA anti-phosphorylcholine antibodies.

27. The method according to any of claims 6-26, or the use according to any of claims 7-26, wherein lower levels of anti-phosphorylcholine antibodies are associated with:
(i) a worse prognosis; and/or
(ii) a more severe disease; and/or
(iii) a higher chance of death.

28. The method according to any of claims 6-27, or the use according to any of claims 7-27, wherein determining the presence and/or amount of anti-phosphorylcholine antibodies in the test sample in Step (b) comprises assessing the presence and/or amount of IgM, IgG and/or IgA anti-phosphorylcholine antibodies.

29. The method according to any of claims 6-28, or the use according to any of claims 7-28, wherein Step (b) comprises determining the presence and/or amount of anti-phosphorylcholine using an immunoassay.

30. The method according to any of claims 6-29, or the use according to any of claims 7-29, wherein the step of predicting the prognosis of the virus infection in the individual in Step (c) comprises calculating whether the amount of anti-phosphorylcholine antibodies is below the 25^{th} percentile and classifying these individuals as more likely to have worse prognosis, a more severe disease and/or a higher chance of death.

31. A method for identifying an individual in need of anti-viral therapy, the method comprising the steps of:
(a) providing a test sample from the individual;
(b) determining the presence and/or amount of anti-phosphorylcholine antibodies in the test sample;
(c) predicting the prognosis of a virus infection in the individual on the basis of the determination in Step (b); and
(d) identifying the individual as one in need of anti-viral therapy on the basis of the determination in Step (c).

32. The method of claim 31, further comprising the step of administering an anti-viral therapy to the individual.

33. A method, use, protein complex or pharmaceutical composition substantially as described herein with reference to the accompanying claims, description, examples and/or figures.


SEQUENCE LISTING (12 sequences)

SEQ ID NO: 1 - DNA, 348 bp, Homo sapiens
  source 1..348: mol_type = genomic DNA
  misc_feature 1..348: note = E01 - VH (Variable domain Heavy Chain)
gaggtgcagctggtggagtccgggggaggcttaattcagcctggggggtccctgagactctcctgtgcagcctctggattcaccttcagtaactactggatgcactgggtccgccaggctccagggaaggggctggtgtgggtctcacgtgttaatagtgacgggacttccacaacctacgcggactccgtgaagggccgattcaccatctccagagacgacgccaagaacacgctgtatctgcaaatgcacagtctgagagccgaggacacggctgtgtattactgtgtggcaactcgcccagataatgactactggggccagggaaccctggtcaccgtctcctca

SEQ ID NO: 2 - protein, 116 aa, Homo sapiens
  REGION 1..116: note = E01 - VH (Variable Heavy Chain)
EVQLVESGGGLIQPGGSLRLSCAASGFTFSNYWMHWVRQAPGKGLVWVSRVNSDGTSTTYADSVKGRFTISRDDAKNTLYLQMHSLRAEDTAVYYCVATRPDNDYWGQGTLVTVSS

SEQ ID NO: 3 - DNA, 327 bp, Homo sapiens
  source 1..327: mol_type = genomic DNA
  misc_feature 1..327: note = E01 - VL (Variable domain Light Chain)
tcagaaattgtgttgacgcagtctccaggcactctgtctttgcctccaggggaaagagccaccctgtcctgcagggccagtcagagtgttagcagcagctccttagcctggtaccggcagaaacctggccaggctcccagactcctcatctatgagacatccagcagggccactggcatcccagacaggttcagtggcagtgggtctgggacagacttcactctcaccatcagcagactggagcctgaagattttgcagtgttttactgtctgcattatggtagctcatcttggacgttcggccaagggaccaaggtggaaatcaaa

SEQ ID NO: 4 - protein, 109 aa, Homo sapiens
  REGION 1..109: note = E01 - VL (Variable domain Light Chain)
SEIVLTQSPGTLSLPPGERATLSCRASQSVSSSSLAWYRQKPGQAPRLLIYETSSRATGIPDRFSGSGSGTDFTLTISRLEPEDFAVFYCLHYGSSSWTFGQGTKVEIK

SEQ ID NO: 5 - DNA, 390 bp, Homo sapiens
  source 1..390: mol_type = genomic DNA
  misc_feature 1..390: note = A01 - VH (Variable domains Heavy Chain)
caggtgcagctgcaggagtcgggcccaggactggtgaagccttcggagaccctgtccctcacctgcactgtctctggtgactccatcagtagtgactactggagttgggtccggcagcccccagggaggggactggagtggattggttacgtctattatagtggggtcaccaggtacaatccctcgctcaacagtcgagtcaccatgttaatagacacgtccaagaaatatttctccctgaagttgaggtctgtgacggccgcagacacggccgtgtattactgtgcgagacattatgagtcctcctacgttaatggtggaggtcagcagcagtcatggccaaactggttcgacgcctggggccacggaattctggtcaccgtctcctca

SEQ ID NO: 6 - protein, 130 aa, Homo sapiens
  REGION 1..130: note = A01 - VH (variable domains) Heavy Chain
QVQLQESGPGLVKPSETLSLTCTVSGDSISSDYWSWVRQPPGRGLEWIGYVYYSGVTRYNPSLNSRVTMLIDTSKKYFSLKLRSVTAADTAVYYCARHYESSYVNGGGQQQSWPNWFDAWGHGILVTVSS

SEQ ID NO: 7 - DNA, 342 bp, Homo sapiens
  source 1..342: mol_type = genomic DNA
  misc_feature 1..342: note = A01 - VL (Variable domain Light Chain)
gacatcgtgatgacccagtctccagactccctggctgtgtctctgggcgagagggccaccatcaactgcaagtccagccaggatattttatacagtcccaacaaaaagaattacttagcttggtaccagcagaaaccagggcggcctcctaagatgctgatttactgggcatctacccgggaatccggggtccctgaccgattcaccggcagcgggtctgcgacagacttcactctcaccatcaccagcctgcaggctgaggatgtggcagtttattattgtcagcagtcatattctacgccgttcactttcggccctgggaccaaagtggatatcaaacgt

SEQ ID NO: 8 - protein, 114 aa, Homo sapiens
  REGION 1..114: note = A01 - VL (Variable domain Light Chain)
DIVMTQSPDSLAVSLGERATINCKSSQDILYSPNKKNYLAWYQQKPGRPPKMLIYWASTRESGVPDRFTGSGSATDFTLTITSLQAEDVAVYYCQQSYSTPFTFGPGTKVDIKR

SEQ ID NO: 9 - DNA, 375 bp, Homo sapiens
  source 1..375: mol_type = genomic DNA
  misc_feature 1..375: note = D05 - VH (Variable domain Heavy Chain
gaggtgcagctgttggagtctgggggaggcttggtacagccgggggggtccctgagactctcctgtgcagcctctggattcacctttagcaattatgccatgacctgggtccgccaggctccagggaaggggctggagtgggtctcaagtatgagtgctagtgatggtggcacatactatgcagactccgtgaagggtcggttcattatttcgagagacaattccaacggcgcgctgtttctgcaaatgaacagcgtgagagtcgaggacacggccatgtatttctgtacgaaagaaatttggcataatagcgattacggtaacccccgggaagagagcttctggggccagggaaccctggtcaccgtctcctca

SEQ ID NO: 10 - protein, 125 aa, Homo sapiens
  REGION 1..125: note = D05 - VH (Variable domain Heavy Chain)
EVQLLESGGGLVQPGGSLRLSCAASGFTFSNYAMTWVRQAPGKGLEWVSSMSASDGGTYYADSVKGRFIISRDNSNGALFLQMNSVRVEDTAMYFCTKEIWHNSDYGNPREESFWGQGTLVTVSS

SEQ ID NO: 11 - DNA, 321 bp, Homo sapiens
  source 1..321: mol_type = genomic DNA
  misc_feature 1..321: note = D05 - VL (Variable domain Light Chain)
gacatccagatgacccagtctccctcctcactgtctgcgtctgtaggagacagagtcgccatcacttgtcgggcgagtcaggacattaaaaattacgtagcctggtttcagcagaaaccagggaaagcccctaagtccctgatctttgctgcatccagtttgcaaggtggggtcccatcaaagttcagcggcagtgcgtctgggacagatttcactctcaccatcagcagcctgcagcctgaagattttgcaacttattactgccaacaatattatagttaccctctcgctttcggcggggggaccaaggtggagatcaaa

SEQ ID NO: 12 - protein, 107 aa, Homo sapiens
  REGION 1..107: note = D05 - VL (Variable domain Light Chain)
DIQMTQSPSSLSASVGDRVAITCRASQDIKNYVAWFQQKPGKAPKSLIFAASSLQGGVPSKFSGSASGTDFTLTISSLQPEDFATYYCQQYYSYPLAFGGGTKVEIK